(19) United States
(12) Patent Application Publication
(10) Pub. No.: US 2025/0340831 A1
(43) Pub. Date: Nov. 6, 2025

(54) DEVICES AND METHODS FOR CELL ENCAPSULATION

(71) Applicant: SATELLITE BIOSCIENCES, INC., Cambridge, MA (US)

(72) Inventors: Amanda CHEN, Cambridge, MA (US); Thomas Jay LOWERY, JR., Sudbury, MA (US); Peter MOUA, Boston, MA (US); Christopher MONTALBANO, Huntington, NY (US); Greg MONTALBANO, Huntington, NY (US); Andrew MARTIN, Glen Cove, NY (US)

(21) Appl. No.: 18/869,172

(22) PCT Filed: May 26, 2023
(86) PCT No.: PCT/US2023/067538

§ 371 Date: Nov. 25, 2024

Related U.S. Application Data
(60) Provisional application No. 63/346,529, filed on May 27, 2022.

(51) Int. Cl. C12N 5/00; C12M 1/00; C12M 1/12; C12M 3/00; C12N 5/077

(52) U.S. Cl. CPC C12N 5/0012; C12M 21/08; C12M 23/38; C12M 25/04; C12N 5/0652; C12N 2533/56

(57) ABSTRACT

Featured is a device for encapsulating cells and aggregates thereof. Also featured are methods of encapsulating aggregates including, e.g., parenchymal cells (e.g., hepatocytes) and stromal cells, in a biocompatible scaffold.

### FIELD OF THE INVENTION

The present invention relates generally to devices for encapsulating cell aggregates in a biocompatible scaffold.

### BACKGROUND OF THE INVENTION

Many diseases result from damage, malfunction, or loss of a single organ or tissue type. While certain strategies such as organ transplants can be effective, the demand for replacement organs is great. Tissue therapeutics, including the development of engineered tissue constructs (e.g., cell-based implants), are among the most promising multidisciplinary approaches to fulfill this demand. However, despite significant advances in the fields of cell biology, microfluidics, and engineering, to date, conventional approaches have failed to re-create functional tissues at a scale necessary to impart therapeutic efficacy. Formation of cell aggregates and grafts containing the same to generate tissues is critical as a first step towards creating useful tissues. However, mimicking biological conditions to use these aggregates to create grafts remains challenging. Accordingly, new devices and methods for forming grafts are needed.

### SUMMARY OF THE INVENTION

In one aspect, the invention features a device that includes a well plate with one or more wells and an indexed mold floor. The indexed mold floor includes one or more protrusions that are configured to insert into the one or more wells and form a bottom surface for the one or more wells.

The well plate may include a plurality of wells. For example, the well plate may include from 1 to 1,000 wells, e.g., from 1 to 10 (e.g., 1, 2, 3, 4, 5, 6, 7, 8, 9, or 10), 10 to 100, (e.g., 20, 30, 40, 50, 60, 70, 80, 90, or 100), or 100 to 1,000 (e.g., 200, 300, 400, 500, 600, 700, 800, 900, or 1,000) wells. In some embodiments, the well plate includes 2, 4, 6, 8, 12, 16, 24, 32, 48, 64, 96, 128, 256, or 384 wells.

The indexed mold floor may include a plurality of protrusions. For example, the indexed mold floor may include from 1 to 1,000 protrusions, e.g., from 1 to 10 (e.g., 1, 2, 3, 4, 5, 6, 7, 8, 9, or 10), 10 to 100 (e.g., 20, 30, 40, 50, 60, 70, 80, 90, or 100), or 100 to 1,000 (e.g., 200, 300, 400, 500, 600, 700, 800, 900, or 1,000) protrusions. In some embodiments, the indexed mold floor includes 2, 4, 6, 8, 12, 16, 24, 32, 48, 64, 96, 128, 256, or 384 protrusions. In some embodiments, the well plate and the indexed mold floor have the same corresponding number of wells and protrusions, respectively.

In some embodiments, the indexed mold floor is slidably connected to the well plate such that the one or more protrusions can move vertically with respect to the one or more wells.

In some embodiments, the indexed mold floor is rigid. In some embodiments, the indexed mold floor is flexible.

In some embodiments, the one or more wells are cylindrical, oval, square, rectangular, pentagonal, hexagonal, heptagonal, or octagonal.

In some embodiments, each well has a diameter of from 1 mm to 500 mm. For example, each well may have a diameter of from 1 mm to 10 mm (e.g., 1 mm, 2 mm, 3 mm, 4 mm, 5 mm, 6 mm, 7 mm, 8 mm, 9 mm, or 10 mm), 10 mm to 100 mm (e.g., 20 mm, 30 mm, 40 mm, 50 mm, 60 mm, 70 mm, 80 mm, 90 mm, or 100 mm), or 100 mm to 500 mm (e.g., 150 mm, 200 mm, 250 mm, 300 mm, 350 mm, 400 mm, 450 mm, or 500 mm).

In some embodiments, the device further includes one or more gaskets, wherein each gasket is configured to form a seal between each protrusion and each well. In some embodiments, the protrusion includes the gasket.

In some embodiments, the device further includes a vessel connected to the well plate and configured to serve as a storage receptacle.

In some embodiments, the vessel includes a tube array housing that includes one or more storage tubes.

In some embodiments, the one or more storage tubes are aligned with the one or more wells. For example, each storage tube may correspond to single well of the well plate.

In some embodiments, each storage tube has a volume of from 1 cm³ to 1,000 cm³. For example, each storage tube may have a volume of from 1 cm³ to 10 cm³ (e.g., 1 cm³, 2 cm³, 3 cm³, 4 cm³, 5 cm³, 6 cm³, 7 cm³, 8 cm³, 9 cm³, or 10 cm³), 10 cm³ to 100 cm³, (e.g., 20 cm³, 30 cm³, 40 cm³, 50cm³, 60 cm³, 70 cm³, 80 cm³, 90 cm³, or 100 cm³), or 100 cm³ to 1,000 cm³ (e.g., 200 cm³, 300 cm³, 400 cm³, 500 cm³, 600 cm³, 700 cm³, 800 cm³, 900 cm³, or 1,000 cm³).

The indexed tube array housing may include a plurality of tubes. For example, the tube array housing may include from 1 to 1,000 tubes, e.g., from 1 to 10 (e.g., 1, 2, 3, 4, 5, 6, 7, 8, 9, or 10), 10 to 100 (e.g., 20, 30, 40, 50, 60, 70, 80, 90, or 100), or 100 to 1,000 (e.g., 200, 300, 400, 500, 600, 700, 800, 900, or 1,000) tubes. In some embodiments, the tube array housing includes 2, 4, 6, 8, 12, 16, 24, 32, 48, 64, 96, 128, 256, or 384 tubes. In some embodiments, the tube array housing and the well plate have the same corresponding number of tubes and wells, respectively.

In some embodiments, the well plate further includes a fluid tray connected to the well plate and configured to prevent fluid overflow. The fluid tray may be integrally connected to the well plate or may be a separate component. The fluid tray may have a height of, e.g., from 0.1 mm to 100 mm above a surface of the well plate. For example, in some embodiments, the fluid tray may have a height of from 0.1 mm to 1 mm (e.g., 0.2 mm, 0.3 mm, 0.4 mm, 0.5 mm, 0.6 mm, 0.7 mm, 0.8 mm, 0.9 mm, or 1 mm), 1 mm to 10 mm (e.g., 2 mm, 3 mm, 4 mm, 5 mm, 6 mm, 7 mm, 8 mm, 9 mm, or 10 mm), or 10 mm to 100 mm (e.g., 20 mm, 30 mm, 40 mm, 50 mm, 60 mm, 70 mm, 80 mm, 90 mm, or 100 mm). In some embodiments, the height of the fluid tray is from 0.1 mm to 10 mm above the surface of the well plate.

In some embodiments, the device further includes a lid configured to cover the well plate. In other embodiments, the device further includes one or more sample identification elements, such as barcodes. The one or more sample identification elements may be present on the lid.

In some embodiments, the device further includes a fastening element that secures the indexed mold floor to the well plate.

In another aspect, the invention features a method of encapsulating a population of aggregates. The method includes the steps of (a) providing a device as described herein, e.g., as described in any of the above embodiments; and (b) introducing a population of aggregates and a biocompatible scaffold into the one or more wells. The method further includes the step of (c) introducing a polymerizing agent into the one or more wells to polymerize the biocompatible scaffold, thereby encapsulating the population of aggregates, e.g., in the biocompatible scaffold. The aggregates may include, for example, two or more populations of cells (e.g., two, three, four, five, six, seven, eight, nine, ten, or more populations of cells). The aggregates may include, for example, a first population of cells and a second population of cells. The first population of cells may include, for example, stromal cells. The second population of cells may include, for example, parenchymal cells.

In some embodiments the first population of cells and/or the second population of cells are induced pluripotent (iPSC)-derived cells, engineered cells, primary cells, embryonic stem cells (ESC)-derived cells, or transdifferentiated cells. In some embodiments, the primary cells include primary cells expanded in vitro.

In some embodiments, the engineered cells are engineered to express or secrete a protein (e.g., an antibody, a cytokine, an enzyme, a coagulation factor, or a hormone). In some embodiments, the protein is an endogenous human protein or an engineered protein.

In some embodiments, the first and/or second population of cells includes endocrine, exocrine, paracrine, heterocrine, autocrine, or juxtacrine cells.

In some embodiments, the first and/or second population of cells includes leading cells, adrenal cortical cells, pituitary cells, thyrocytes, granulosa cells, mammary gland epithelial cells, thymocytes, thymic epithelial cells, hypothalamus cells, skeletal muscle cells, smooth muscle cells, and/or neuronal cells.

In some embodiments, the pituitary cells include thyrotropic pituitary cells, lactotropic pituitary cells, corticotropic pituitary cells, somatotropic pituitary cells, and/or gonadotropic pituitary cells. In some embodiments, the neuronal cells include dopaminergic cells.

In some embodiments, the first and/or second population of cells includes parenchymal cells (e.g., hepatocytes, pancreatic exocrine cells, myocytes, pancreatic endocrine cells, neurons, enterocytes, adipocytes, splenic cells, kidney cells, biliary cells, Kupffer cells, stellate cells, cardiac muscle cells, alveolar cells, bronchiolar cells, club cells, urothelial cells, mucous cells, parietal cells, chief cells, G cells, goblet cells, enteroendocrine cells, Paneth cells, M cells, tuft cells, glial cells, gall bladder cells, keratinocytes, melanocytes, Merkel cells, Langerhans cells, osteocytes, osteoclasts, esophageal cells, photoreceptor cells, and corneal epithelial cells). In some embodiments, the parenchymal cells are pancreatic cells (e.g., alpha, beta, gamma, delta, epsilon cells, or any combination thereof). In some embodiments, the parenchymal cells include beta cells.

In some embodiments, the first and/or second population of cells are engineered cells, primary cells, or transdifferentiated cells.

In some embodiments, the method includes encapsulating two or more populations of cells (e.g., two, three, four, five, six, seven, eight, nine, ten, or more populations of cells).

The introduction of aggregates of the first population of cells and the second population of cells can be sequential, e.g., the aggregates of the first population of cells may be first introduced, e.g., in a first medium, and the aggregates of the second population of cells may be subsequently introduced, e.g., in a second medium. Similarly, the aggregates of second population cells may be first introduced, e.g., in a first medium, and the aggregates of the first population cells may be subsequently introduced, e.g., in a second medium. Alternatively, the aggregates of the first population of cells and the aggregates of second population of cells may be introduced in the same medium.

The introduction of aggregates of stromal cells and parenchymal cells can be sequential, e.g., the aggregates of stromal cells may be first introduced, e.g., in a first medium, and the aggregates of parenchymal cells may be subsequently introduced, e.g., in a second medium. Similarly, the aggregates of parenchymal cells may be first introduced, e.g., in a first medium, and the aggregates of stromal cells may be subsequently introduced, e.g., in a second medium. Alternatively, the aggregates of stromal cells and the aggregates of parenchymal cells may be introduced in the same medium. In some embodiments, the aggregates include a combination of stromal cells and parenchymal cells.

In some embodiments, the biocompatible scaffold includes fibrin.

In some embodiments, the biocompatible scaffold further includes a reinforcing agent. For example, the reinforcing agent may include collagen, poly(ethylene glycol), polyvinylidene acetate (PVDA), polyvinylidene fluoride (PVDF), poly(lactic-co-glycolic) acid (PLGA), or poly (l-lactic acid) (PLLA).

In some embodiments, the polymerizing agent is or includes thrombin.

In some embodiments, the device further includes a vessel connected to the well plate and configured to serve as a storage receptacle. The vessel may further include a storage media.

In some embodiments, the method further includes raising the indexed mold floor to eject the encapsulated population of aggregates from the one or more wells.

In some embodiments, the method further includes distributing the encapsulated population of aggregates from the one or more wells into the one or more storage tubes.

In some embodiments, the encapsulated population of aggregates is distributed by gravity.

In some embodiments, the first population of cells comprises stromal cells and the second population of cells comprises parenchymal cells. In some embodiments, the parenchymal cells are hepatocytes, pancreatic exocrine cells, myocytes, pancreatic endocrine cells, neurons, enterocytes, adipocytes, splenic cells, kidney cells, biliary cells, Kupffer cells, stellate cells, cardiac muscle cells, alveolar cells, bronchiolar cells, club cells, urothelial cells, mucous cells, parietal cells, chief cells, G cells, goblet cells, enteroendocrine cells, Paneth cells, M cells, tuft cells, glial cells, gall bladder cells, keratinocytes, melanocytes, Merkel cells, Langerhans cells, osteocytes, osteoclasts, esophageal cells, photoreceptor cells, or corneal epithelial cells. In some embodiments, the parenchymal cells are pancreatic cells (e.g., alpha, beta, gamma, delta, epsilon cells, or any combination thereof).

In some embodiments, the hepatocytes are primary human hepatocytes.

In some embodiments, the stromal cells are fibroblasts, endothelial cells, or pericytes.

In some embodiments, the fibroblasts are normal human dermal fibroblasts or neonatal foreskin fibroblasts. In some embodiments, the fibroblasts are normal human dermal fibroblasts.

In another aspect, the invention features an encapsulated population of aggregates of a first population of cells and a second population of cells produced by a method as described herein. In some embodiments, the first population of cells comprises stromal cells and the second population of cells comprises parenchymal cells.

In some embodiments, the encapsulated population of aggregates is suitable for implantation into a subject (e.g., a human subject).

In another aspect, the invention features a system that includes a well plate that includes one or more wells and an indexed mold floor. The indexed mold floor includes one or more protrusions that are configured to insert into the one or more wells and form a bottom surface for the one or more wells.

In some embodiments, the system further includes a vessel. The vessel may include a tube array housing with one or more storage tubes. The storage tubes may be aligned with the one or more wells.

In some embodiments, the system further includes a lid configured to cover the well plate.

In some embodiments, the system further includes one or more sample identification elements, such as barcodes. The one or more sample identification elements may be present on the lid.

In another aspect, the invention features a kit that includes a well plate that includes one or more wells and an indexed mold floor. The indexed mold floor includes one or more protrusions that are configured to insert into the one or more wells and form a bottom surface for the one or more wells.

In some embodiments, the kit further includes a vessel. The vessel may include a tube array housing with one or more storage tubes. The storage tubes maybe aligned with the one or more wells.

In some embodiments, the kit further includes a lid configured to cover the well plate.

In some embodiments, the kit further includes one or more sample identification elements, such as barcodes. The one or more sample identification elements may be present on the lid.

In some embodiments, the kit further includes one or more of a population of aggregates that includes a first population of cells and a second population of cells, a biocompatible scaffold, and a polymerizing agent. In some embodiments, the first population of cells comprises stromal cells and the second population of cells comprises parenchymal cells.

### BRIEF DESCRIPTION OF THE DRAWINGS

FIG. 1 is a schematic drawing showing a device with a well plate (1) with wells (2), an indexed mold floor (3) with protrusions (4), and a fluid tray (9). The wells (2) are sealed with gaskets (5).

FIG. 2 is a schematic drawing showing a device with a well plate (1) with wells (2), an indexed mold floor (3) with protrusions (4), and a fluid tray (9). The well plate includes a population of aggregates and a biocompatible scaffold in wells (2), which are sealed with gaskets (5).

FIG. 3 is a schematic drawing showing a device with a well plate (1) with wells (2), an indexed mold floor (3) with protrusions (4), and a fluid tray (9). The well plate includes a population of aggregates and a biocompatible scaffold in wells (2), which are sealed with gaskets (5). The indexed mold floor (3) is pushed upwards to release the population of aggregates and the biocompatible scaffold from wells (2)

FIG. 4 is a schematic drawing of a device that includes a well plate (1), a fluid tray (9), and a lid (10). The lid contains sample identification elements (11), such as barcodes, above each well (2).

FIG. 5 is a schematic drawing of a device that includes a well plate (1) with wells (2). The device is shown with protrusions (4) of an indexed mold floor and a fluid tray (9).

FIG. 6 is a schematic drawing of a device that includes a well plate (1) with wells (2) and a fluid tray (9). A population of aggregates and a biocompatible scaffold is shown in wells (2).

FIG. 7 is a cross-sectional view of a device that includes a well plate (1) with wells (2), an indexed mold floor (3) with protrusions (4), and gaskets (5). A population of aggregates and a biocompatible scaffold is shown in wells (2).

FIG. 8 is a schematic drawing of a device that includes a storage vessel (6) with a tube array housing (7) containing storage tubes (8).

FIG. 9 is a schematic drawing showing a device with a well plate (1) with wells (2), an indexed mold floor (3), a fluid tray (9), and a vessel (6) that includes a tube array housing (7).

FIG. 10 is a cross-sectional view of the device in FIG. 9 showing a device with a well plate (1) with wells (2), an indexed mold floor (3) with protrusions (4), a fluid tray (9), and vessel (6) that includes a tube array housing (7) that includes storage tubes (8). The wells (2) are sealed with gaskets (5).

FIG. 11 is a schematic drawing as in FIG. 10 with the protrusions (4) of indexed mold floor (3) pushed into the wells (2) of well plate (1).

FIG. 12 is a schematic drawing showing a device with a well plate (1), an indexed mold floor (3) with protrusions (4). The device includes a fastening element (12) that secures the indexed mold floor to the well plate (1).

FIG. 13 is a schematic drawing showing a device as described herein with exemplary dimensions (mm).

FIG. 14 is a schematic drawing showing a device as described herein with a fastening element (12) that secures the indexed mold floor (3) to the well plate (1).

FIG. 15 is an exploded view of the device of FIG. 14.

FIG. 16 is a photograph of encapsulated aggregates produced by a prototype encapsulation device.

### DEFINITIONS

Cells can be from established cell lines, or they can be primary cells, where “primary cells,” “primary cell lines,” and “primary cultures” are used interchangeably herein to refer to cells and cells cultures that have been derived from an individual (e.g., a human individual) and allowed to grow in vitro for a limited number of passages, e.g., splitting, of the culture. For example, primary cultures can be cultures that have been passaged 0 times, 1 time, 2 times, 4 times, 5 times, 10 times, or 15 times, but not enough times go through the crisis stage. Primary cell lines can be maintained for fewer than 10 passages in vitro. If the cells are primary cells, such cells can be harvested from an individual by any convenient method. For example, cells from tissues such as skin, muscle, bone marrow, spleen, liver, pancreas, lung, intestine, stomach, or other tissues are most conveniently harvested by biopsy. An appropriate solution can be used for dispersion or suspension of the harvested cells. Such solution will generally be a balanced salt solution, e.g., normal saline, phosphate-buffered saline (PBS), Hank's balanced salt solution, and the like, conveniently supplemented with fetal calf serum or other naturally occurring factors, in conjunction with an acceptable buffer at low concentration, generally from 5-25 mM. Convenient buffers include HEPES (4-(2-hydroxyethyl)-1-piperazineethanesulfonic acid), phosphate buffers, lactate buffers, and the like. The cells can be used immediately, or they can be stored, frozen, for long periods of time, being thawed and capable of being reused. In such cases, the cells will usually be frozen in 10% dimethyl sulfoxide (DMSO), 50% serum, 40% buffered medium, or some other such solution as is commonly used in the art to preserve cells at such freezing temperatures and thawed in a manner as commonly known in the art for thawing frozen cultured cells. For example, hepatocytes may be isolated by conventional methods (Berry and Friend, 1969, J. Cell Biol. 43:506-520) which can be adapted for human liver biopsy or autopsy material (e.g., to garner primary human hepatocytes).

As used herein, the term “cell type” refers to a group of cells sharing a phenotype that is statistically separable based on gene expression data. For example, cells of a common cell type may share similar structural and/or functional characteristics, such as similar gene activation patterns and antigen presentation profiles. Cells of a common cell type may include those that are isolated from a common tissue (e.g., epithelial tissue, neural tissue, connective tissue, or muscle tissue) and/or those that are isolated from a common organ, tissue system, blood vessel, or other structure and/or region in an organism.

As used herein, the term “parenchymal cells” refers to cells of, or derived from, the parenchyma of an organ or gland, e.g., a mammalian organ or gland. The parenchyma of an organ or gland is the functional tissue of the organ or gland, as distinguished from surrounding or supporting or connective tissue. As such, parenchymal cells are attributed with carrying out the particular function, or functions, of the organ or gland, often referred to in the art as “tissue-specific” function. Parenchymal cells include, but are not limited to, hepatocytes, pancreatic cells (e.g., alpha, beta, gamma, delta, and epsilon cells), myocytes, e.g., smooth muscle cells, cardiac myocytes, and the like, enterocytes, renal epithelial cells and other kidney cells, brain cell (e.g., neurons or glial cells, e.g., astrocytes), respiratory epithelial cells, stem cells, and blood cells (e.g., erythrocytes and lymphocytes), adult and embryonic stem cells, blood-brain barrier cells, adipocytes, splenocytes, osteoblasts, osteoclasts, and other parenchymal cell types known in the art. Because parenchymal cells are responsible for tissue-specific function, parenchymal cells express or secrete certain tissue specific markers.

Certain precursor cells can also be included as “parenchymal cells,” in particular, if they are committed to becoming the more differentiated cells described above, for example, liver progenitor cells, oval cells, adipocytes, osteoblasts, osteoclasts, myoblasts, stem cells (e.g., embryonic stem cells, hematopoietic stem cells, mesenchymal stem cells, or endothelial stem cells), and the like. In some embodiments, stem cells can be encapsulated and/or implanted under specified conditions such that they are induced to differentiate into a desired parenchymal cell type, for example, in the construct and/or in vivo. It is also contemplated that parenchymal cells derived from cell lines can be used in the methodologies of the invention.

### DETAILED DESCRIPTION

The present invention features, inter alia, devices, methods, systems, and kits for encapsulating cell aggregates in a biocompatible scaffold. Such devices may be used to form tissue grafts containing cell aggregates. Formation of cell aggregates for tissue engineering is critical as a first step towards creating useful tissues for various therapeutic approaches. Once the aggregates are formed, they typically must be encapsulated in a suitable scaffold to form a biocompatible graft. However, mimicking biological conditions to achieve such encapsulation, scaling up encapsulation, and minimizing variation between grafts has been challenging to overcome. Moreover, once grafts are generated, manipulating and packaging them for downstream uses without imparting any damage is important.

The present invention solves this problem by providing a device that includes a well plate with one or more wells and an indexed mold floor. The indexed mold floor includes protrusions that are configured to insert into the wells and form a bottom surface for the wells. The aggregates and a biocompatible scaffold can be deposited into the wells, while a polymerizing agent can be added to polymerize the scaffold to encapsulate the aggregates within the scaffold. The device may further include a fluid tray for supplying the encapsulated aggregates with necessary media to control polymerization and encapsulation. Following encapsulation of the aggregates, the indexed mold floor can push out the graft of encapsulated aggregates for facile collection and downstream use.

### Devices

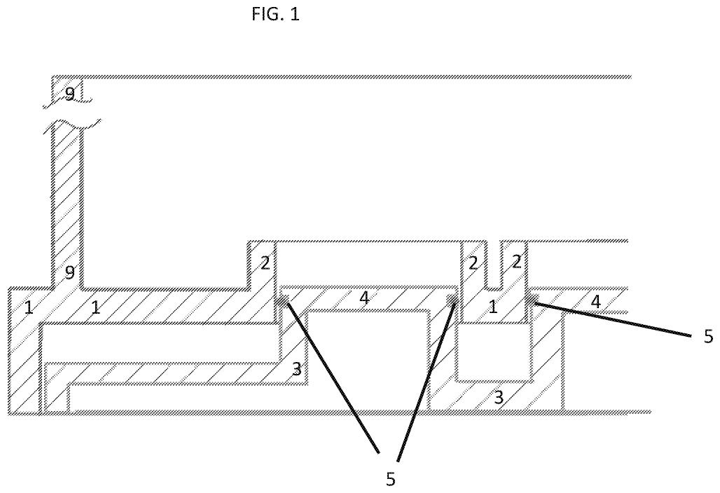

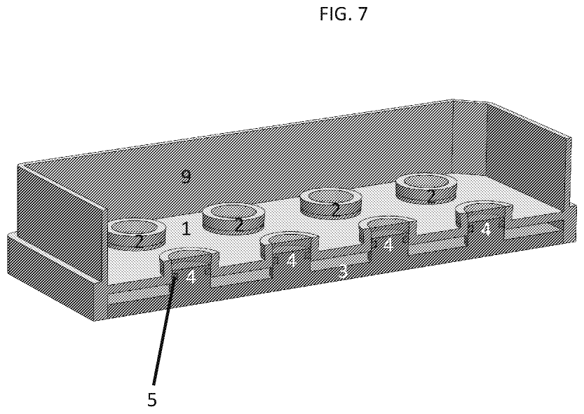

The devices described herein include a plate device that includes a well plate (1) with one or more wells (2) and an indexed mold floor (3). The indexed mold floor includes one or more protrusions (4) that are configured to insert into (e.g., mate with) the one or more wells and form a bottom surface for the one or more wells (see, e.g., FIGS. 1 and 7).

### Well Plate (1)

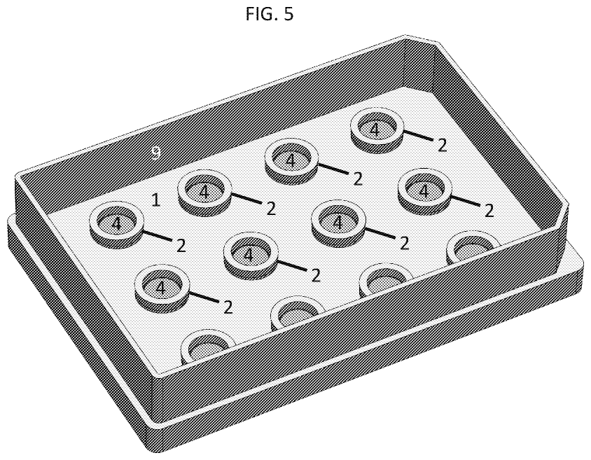

The well plate (1) includes or more wells (2). The well plate (1) may include a plurality of wells (2). For example, the well plate (1) may include from 1 to 1,000 wells, e.g., from 1 to 10 (e.g., 1, 2, 3, 4, 5, 6, 7, 8, 9, or 10), 10 to 100 (e.g., 20, 30, 40, 50, 60, 70, 80, 90, or 100), or 100 to 1,000 (e.g., 200, 300, 400, 500, 600, 700, 800, 900, or 1,000) wells. In some embodiments, the well plate includes 2, 4, 6, 8, 12, 16, 24, 32, 48, 64, 96, 128, 256, or 384 wells. In some embodiments, the well plate includes 12 wells (see, e.g., FIG. 5).

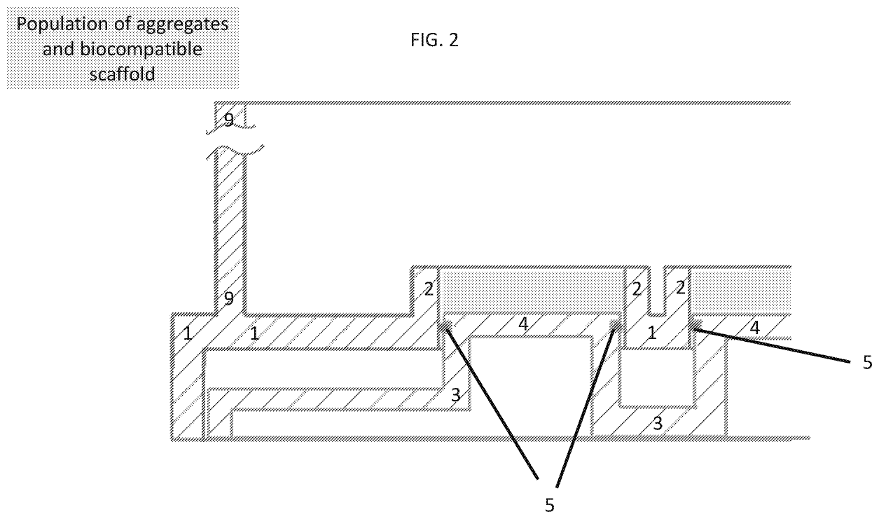

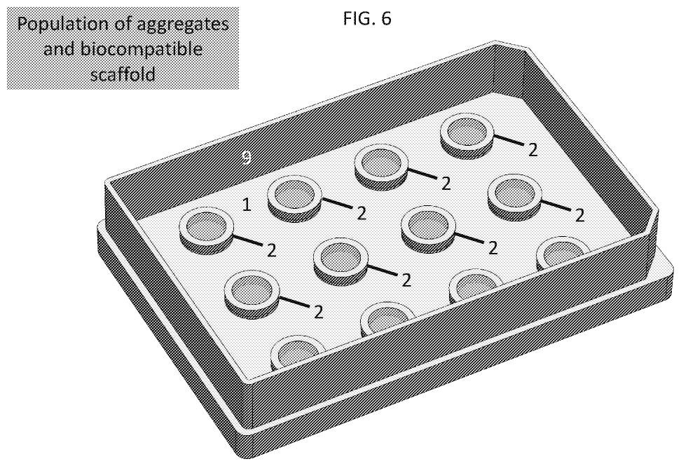

The wells (2) may have any suitable shape or geometry to receive components for making an encapsulated graft of cells (see, e.g., FIGS. 2 and 6). For example, the one or more wells may be, e.g., cylindrical, oval, square, rectangular, pentagonal, hexagonal, heptagonal, or octagonal (e.g., having a cross-section of a shape as described herein).

In some embodiments, each well has a diameter of from 1 mm to 500 mm. For example, each well (2) may have a diameter of from 1 mm to 10 mm (e.g., 1 mm, 2 mm, 3 mm, 4 mm, 5 mm, 6 mm, 7 mm, 8 mm, 9 mm, or 10 mm), 10 mm to 100 mm (e.g., 20 mm, 30 mm, 40 mm, 50 mm, 60 mm, 70 mm, 80 mm, 90 mm, or 100 mm), or 100 mm to 500 mm (e.g., 150 mm, 200 mm, 250 mm, 300 mm, 350 mm, 400 mm, 450 mm, or 500 mm).

In some embodiments, each well has a depth of from 0.1 mm to 500 mm. For example, each well (2) may have a depth of from 0.1 mm to 1 mm (e.g., 0.1 mm, 0.2 mm, 0.3 mm, 0.4 mm, 0.5 mm, 0.6 mm, 0.7 mm, 0.8 mm, 0.9 mm, or 1 mm), 1 mm to 10 mm (e.g., 1 mm, 2 mm, 3 mm, 4 mm, 5 mm, 6 mm, 7 mm, 8 mm, 9 mm, or 10 mm), 10 mm to 100 mm (e.g., 20 mm, 30 mm, 40 mm, 50 mm, 60 mm, 70 mm, 80 mm, 90 mm, or 100 mm), or 100 mm to 500 mm (e.g., 150 mm, 200 mm, 250 mm, 300 mm, 350 mm, 400 mm, 450 mm, or 500 mm).

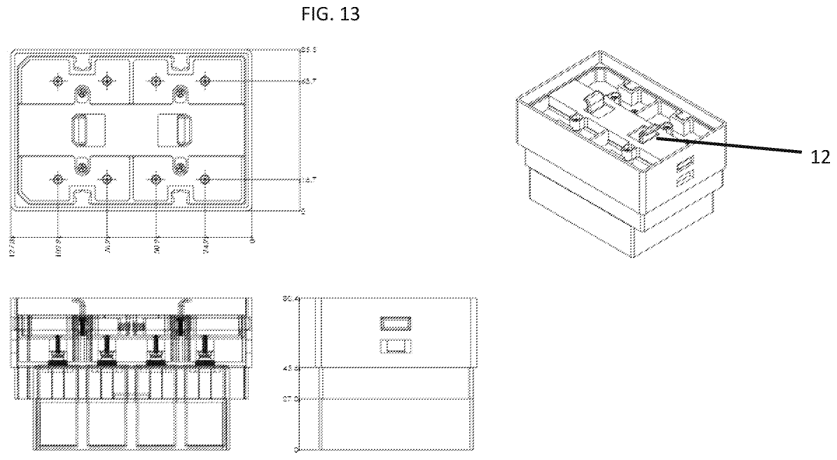

In some embodiments, the length and/or width of the well plate (1) is, independently from 1 cm to about 100 cm. For example, the length and/or width of the well plate (1) may be, independently 1 cm to 10 cm (e.g., 1 cm, 2 cm, 3 cm, 4 cm, 5 cm, 6 cm, 7 cm, 8 cm, 9 cm, or 10 cm), 10 cm to 20 cm (e.g., 11 cm, 12 cm, 13 cm, 14 cm, 15 cm, 16 cm, 17 cm, 18 cm, 19 cm, or 20 cm), 20 cm to 50 cm (e.g., 25 cm, 30 cm, 35 cm, 40 cm, 45 cm, or 50 cm), or 50 cm to 100 cm (e.g., 60 cm, 70 cm, 80 cm, 90 cm, or 100 cm). The well plate (1) may be, e.g., a square or rectangular in shape. Exemplary dimensions are shown in FIG. 13.

In some embodiments, the well plate (1) has a surface area of from 100 mm² to 100,000 mm². For example, the plate (1) may have a surface area of 100 mm² to 1,000 mm² (e.g., 100 mm², 200 mm², 300 mm², 400 mm², 500 mm², 600 mm², 700 mm², 800 mm², 900 mm², or 1,000 mm²), 1,000 mm² to 10,000 mm² (e.g., 2,000 mm², 3,000 mm², 4,000 mm², 5,000 mm², 6,000 mm², 7,000 mm², 8,000 mm², 9,000 mm², or 10,000 mm²), or 10,000 mm² to 100,000 mm² (e.g., 20,000 mm², 30,000 mm², 40,000 mm², 50,000 mm², 60,000 mm², 70,000 mm², 80,000 mm², 90,000 mm², or 100,000 mm²). In some embodiments, the plate (1) has a surface area of 200 mm² to 10,000 mm². In some embodiments, the well plate (1) has a surface area of 300 mm² to 10,000 mm². In some embodiments, the well plate (1) has a surface area of 400 mm² to 1,000 mm². In some embodiments, the well plate (1) has a surface area of 500 mm².

The well plate (1) may include or be composed of polymeric materials, such as polyethylene or polyethylene derivatives, such as cyclic olefin copolymers (COC), polymethylmethacrylate (PMMA), polydimethylsiloxane (PDMS), polycarbonate, polystyrene, polypropylene, polyvinyl chloride, polytetrafluoroethylene, polyoxymethylene, polyether ether ketone, polycarbonate, polystyrene, liquid silicone rubber (e.g., ELASTOSIL® LR 3003/30 A/B), or the like. The well plate (1) may be fabricated in whole or in part from inorganic materials, such as silicon, or other silica-based materials, e.g., glass, quartz, fused silica, borosilicate glass, metals, ceramics, and combinations thereof. Polymeric components may be fabricated using any of a number of processes including soft lithography, embossing techniques, micromachining, e.g., laser machining, or injection molding of the well plate components. In some embodiments, the well plate (1) is rigid. In some embodiments, the well plate is flexible.

### Indexed Mold Floor (3)

The indexed mold (3) floor may include a plurality of protrusions (4). For example, the indexed mold floor (3) may include from 1 to 1,000 protrusions, e.g., from 1 to 10 (e.g., 1, 2, 3, 4, 5, 6, 7, 8, 9, or 10), 10 to 100 (e.g., 20, 30, 40, 50, 60, 70, 80, 90, or 100), or 100 to 1,000 (e.g., 200, 300, 400, 500, 600, 700, 800, 900, or 1,000) protrusions. In some embodiments, the indexed mold floor includes 2, 4, 6, 8, 12, 16, 24, 32, 48, 64, 96, 128, 256, or 384 protrusions. In some embodiments, the indexed mold floor includes 12 protrusions.

The well plate (1) and the indexed mold floor (3) may have the same corresponding number of wells and protrusions, respectively.

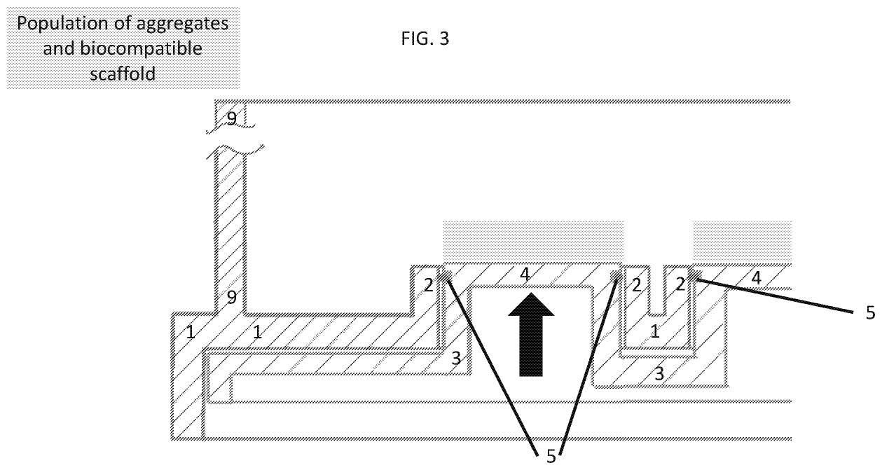

In some embodiments, the indexed mold floor (3) is slidably connected to the well plate such that the one or more protrusions (4) can move vertically with respect to the one or more wells (2). Such a slidable connection allows ejection of the contents of a well (1) upon vertical movement of the protrusions (4) of the indexed mold floor (3) (see, e.g., FIG. 3).

The protrusion (4) of the indexed mold floor (3) may have any suitable shape or geometry to mate with a corresponding well (2) of the well plate (1). For example, the one or more protrusions may be, e.g., cylindrical, oval, square, rectangular, pentagonal, hexagonal, heptagonal, or octagonal (e.g., having a cross-section of a shape as described herein).

In some embodiments, each protrusion has a diameter of from 1 mm to 500 mm. For example, each protrusion may have a diameter of from 1 mm to 10 mm (e.g., 1 mm, 2 mm, 3 mm, 4 mm, 5 mm, 6 mm, 7 mm, 8 mm, 9 mm, or 10 mm), 10 mm to 100 mm (e.g., 20 mm, 30 mm, 40 mm, 50 mm, 60 mm, 70 mm, 80 mm, 90 mm, or 100 mm), or 100 mm to 500 mm (e.g., 150 mm, 200 mm, 250 mm, 300 mm, 350 mm, 400 mm, 450 mm, or 500 mm).

In some embodiments, each protrusion has a length (e.g., a vertical length) of from 0.1 mm to 500 mm. For example, each protrusion (4) may have a length of from 0.1 mm to 1 mm (e.g., 0.1 mm, 0.2 mm, 0.3 mm, 0.4 mm, 0.5 mm, 0.6 mm, 0.7 mm, 0.8 mm, 0.9 mm, or 1 mm), 1 mm to 10 mm (e.g., 1 mm, 2 mm, 3 mm, 4 mm, 5 mm, 6 mm, 7 mm, 8 mm, 9 mm, or 10 mm), 10 mm to 100 mm (e.g., 20 mm, 30 mm, 40 mm, 50 mm, 60 mm, 70 mm, 80 mm, 90 mm, or 100 mm), or 100 mm to 500 mm (e.g., 150 mm, 200 mm, 250 mm, 300 mm, 350 mm, 400 mm, 450 mm, or 500 mm).

In some embodiments, the length of each protrusion (4) corresponds to the depth of each corresponding well (2).

In some embodiments, the length and/or width of the indexed mold floor (3) is, independently from 1 cm to about 100 cm. For example, the length and/or width of the indexed mold floor (3) may be, independently 1 cm to 10 cm (e.g., 1 cm, 2 cm, 3 cm, 4 cm, 5 cm, 6 cm, 7 cm, 8 cm, 9 cm, or 10 cm), 10 cm to 20 cm (e.g., 11 cm, 12 cm, 13 cm, 14 cm, 15 cm, 16 cm, 17 cm, 18 cm, 19 cm, or 20 cm), 20 cm to 50 cm (e.g., 25 cm, 30 cm, 35 cm, 40 cm, 45 cm, or 50 cm), or 50 cm to 100 cm (e.g., 60 cm, 70 cm, 80 cm, 90 cm, or 100 cm). The indexed mold floor (3) may be, e.g., a square or rectangular in shape.

In some embodiments, the indexed mold floor (3) has a surface area of from 100 mm² to 100,000 mm². For example, the plate (1) may have a surface area of 100 mm² to 1,000 mm² (e.g., 100 mm², 200 mm², 300 mm², 400 mm², 500 mm², 600 mm², 700 mm², 800 mm², 900 mm², or 1,000 mm²), 1,000 mm² to 10,000 mm² (e.g., 2,000 mm², 3,000 mm², 4,000 mm², 5,000 mm², 6,000 mm², 7,000 mm², 8,000 mm², 9,000 mm², or 10,000 mm²), or 10,000 mm² to 100,000 mm² (e.g., 20,000 mm², 30,000 mm², 40,000 mm², 50,000 mm², 60,000 mm², 70,000 mm², 80,000 mm², 90,000 mm², or 100,000 mm²). In some embodiments, the plate (1) has a surface area of 200 mm² to 10,000 mm². In some embodiments, the indexed mold floor (3) has a surface area of 300 mm² to 10,000 mm². In some embodiments, the indexed mold floor (3) has a surface area of 400 mm² to 1,000 mm². In some embodiments, the indexed mold floor (3) has a surface area of 500 mm².

The indexed mold floor (3) may include or be composed of polymeric materials, such as polyethylene or polyethylene derivatives, such as cyclic olefin copolymers (COC), polymethylmethacrylate (PMMA), polydimethylsiloxane (PDMS), polycarbonate, polystyrene, polypropylene, polyvinyl chloride, polytetrafluoroethylene, polyoxymethylene, polyether ether ketone, polycarbonate, polystyrene, liquid silicone rubber (e.g., ELASTOSIL® LR 3003/30 A/B), or the like. The indexed mold floor (3) may be fabricated in whole or in part from inorganic materials, such as silicon, or other silica-based materials, e.g., glass, quartz, fused silica, borosilicate glass, metals, ceramics, and combinations thereof. Polymeric components may be fabricated using any of a number of processes including soft lithography, embossing techniques, micromachining, e.g., laser machining, or injection molding of the well plate components. In some embodiments, the indexed mold floor (3) is rigid. In some embodiments, the indexed mold floor is flexible.

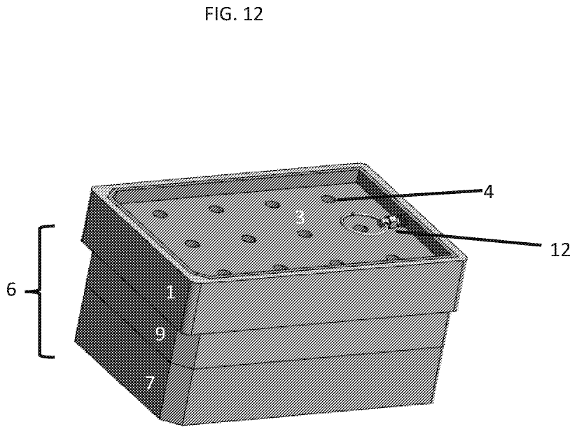

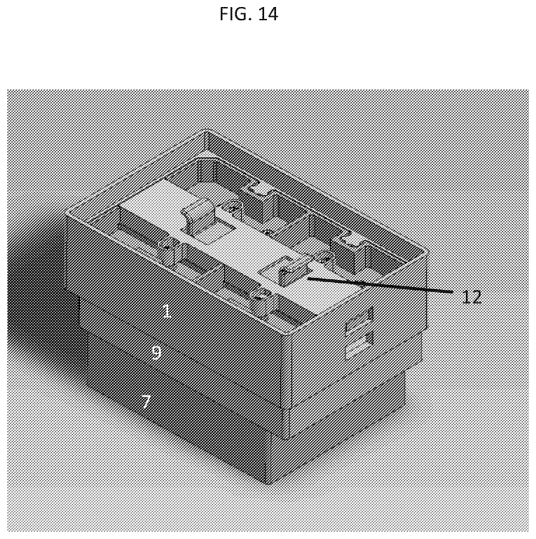

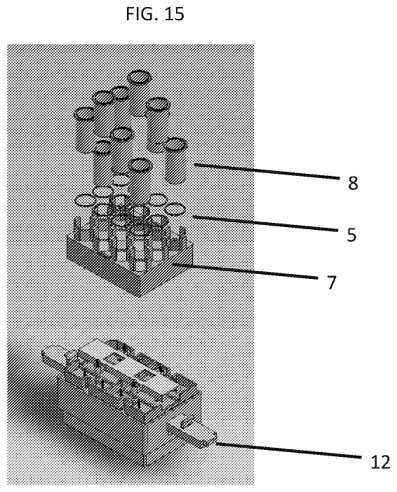

The device may further include a fastening element (12) that secures the indexed mold floor (3) to the well plate (1) (see, e.g., FIGS. 12, 14, and 15). The fastening element may contact the indexed mold floor (3) and the well (1) directly or indirectly. The fastening element may be a key and ring element (FIG. 12) or may include a protrusion through a notch (FIGS. 14 and 15).

### Fluid Tray (9)

The devices described herein may further include a fluid tray (9). The fluid tray (9) may be connected to the well plate (1) and configured to prevent fluid overflow (see, e.g., FIG. 5). The fluid tray may be integrally connected to the well plate or may be a separate component (see, e.g., FIG. 1). The fluid tray may be used to supply the grafts with fluid or media and prevent overflow from the well plate (1). The fluid tray may ensure that the grafts remain submerged in a suitable media.

The fluid tray may have a height of, e.g., from 0.1 mm to 100 mm above a surface of the well plate. For example, in some embodiments, the fluid tray may have a height of from 0.1 mm to 1 mm (e.g., 0.2 mm, 0.3 mm, 0.4 mm, 0.5 mm, 0.6 mm, 0.7 mm, 0.8 mm, 0.9 mm, or 1 mm), 1 mm to 10 mm (e.g., 2 mm, 3 mm, 4 mm, 5 mm, 6 mm, 7 mm, 8 mm, 9 mm, or 10 mm), or 10 mm to 100 mm (e.g., 20 mm, 30 mm, 40 mm, 50 mm, 60 mm, 70 mm, 80 mm, 90 mm, or 100 mm). In some embodiments, the height of the fluid tray is from 0.1 mm to 10 mm above the surface of the well plate.

In some embodiments, the length and/or width of the fluid tray (9) is, independently from 1 cm to about 100 cm. For example, the length and/or width of the fluid tray (9) may be, independently 1 cm to 10 cm (e.g., 1 cm, 2 cm, 3 cm, 4 cm, 5 cm, 6 cm, 7 cm, 8 cm, 9 cm, or 10 cm), 10 cm to 20 cm (e.g., 11 cm, 12 cm, 13 cm, 14 cm, 15 cm, 16 cm, 17 cm, 18 cm, 19 cm, or 20 cm), 20 cm to 50 cm (e.g., 25 cm, 30 cm, 35 cm, 40 cm, 45 cm, or 50 cm), or 50 cm to 100 cm (e.g., 60 cm, 70 cm, 80 cm, 90 cm, or 100 cm). The fluid tray (9) may be, e.g., a square or rectangular in shape.

### Gasket (5)

The devices described herein may include a gasket (5), such as an O-ring. The gasket (5) may be disposed between the protrusions (4) of the indexed mold floor (3) and the wells (2) of the well plate (1) (see, e.g., FIG. 1). The protrusion may include the gasket (5). The gasket (5) may be composed of any suitable material to form a liquid tight seal of the wells (2). For example, the gasket (e.g., O-ring) may include, e.g., a polymer, e.g., a silicone (e.g., silicone rubbers, e.g., PDMS or ELASTOSIL®), fluorosilicone, FKM, FFKM, COC elastomer, etc. The gasket may include an elastomeric polymer, e.g., to allow the gasket to be compressible. A gasket may be a composite of compressible and incompressible materials, e.g., an elastomeric polymer bonded to a non-elastomeric polymer. Gaskets may include thermoset or thermoplastic polymers, or a combination thereof. A gasket may be coated, e.g., to include a one-sided adhesive, a double-sided adhesive, a polymer coating, or a hydrophobic coating. A hydrophobic coating on the gasket may act to improve sealing (e.g., to prevent leaks), and/or to reduce adhesion from about the gasket, the wells (2), and the protrusions (4), e.g., to allow for easier removal.

The device may include a plurality of gaskets (5) (e.g., O-rings). For example, the device may include from 1 to 1,000 gaskets, e.g., from 1 to 10 (e.g., 1, 2, 3, 4, 5, 6, 7, 8, 9, or 10), 10 to 100 (e.g., 20, 30, 40, 50, 60, 70, 80, 90, or 100), or 100 to 1,000 (e.g., 200, 300, 400, 500, 600, 700, 800, 900, or 1,000) gaskets. In some embodiments, the device includes 2, 4, 6, 8, 12, 16, 24, 32, 48, 64, 96, 128, 256, or 384 gaskets. In some embodiments, the device includes 12 gaskets. The device may include the same corresponding number of wells (2) and gaskets (5). The device may include the same corresponding number of wells (2), protrusions (4), and gaskets (5).

### Vessel (6)

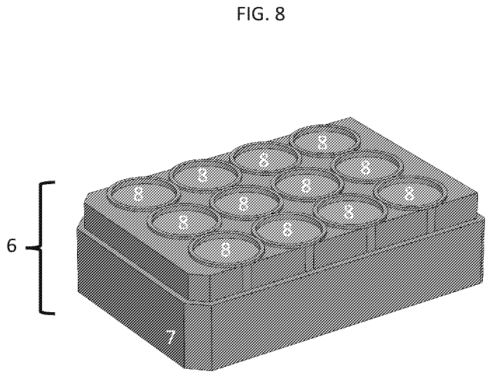

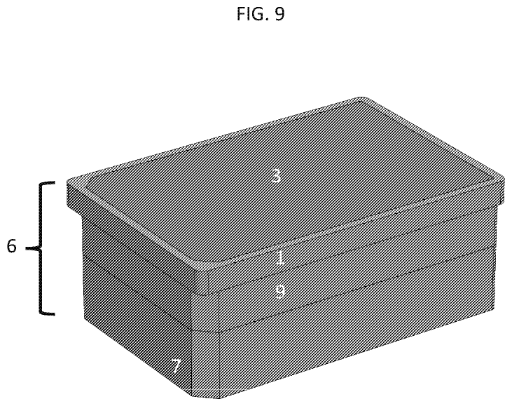

The devices described herein may further include a vessel (6). The vessel (6) may be connected to the well plate (1) to serve as a storage receptacle, e.g., for a graft, e.g., following release from the wells (2). In some embodiments, the vessel (6) includes a tube array housing (7) that includes one or more storage tubes (8) (see, e.g., FIGS. 8 and 9). The storage tubes (8) may include a storage media to store the grafts.

In some embodiments, the one or more storage tubes (8) are aligned with the one or more wells. For example, each storage tube may correspond to a single well of the well plate.

In some embodiments, each storage tube has a volume of from 1 cm³ to 1,000 cm³. For example, each storage tube may have a volume of from 1 cm³ to 10 cm³ (e.g., 1 cm³, 2 cm³, 3 cm³, 4 cm³, 5 cm³, 6 cm³, 7 cm³, 8 cm³, 9 cm³, or 10 cm³), from 10 cm³ to 100 cm³ (e.g., 20 cm³, 30 cm³, 40 cm³, 50 cm³, 60 cm³, 70 cm³, 80 cm³, 90 cm³, or 100 cm³), or 100 cm³ to 1,000 cm³ (e.g., 200 cm³, 300 cm³, 400 cm³, 500 cm³, 600 cm³, 700 cm³, 800 cm³, 900 cm³, or 1,000 cm³).

The vessel (6) and/or the storage tubes (8) may have any suitable shape or geometry to be compatible with a device as described herein. For example, the vessel (6) and/or storage tubes (7) may be, e.g., cylindrical, oval, square, rectangular, pentagonal, hexagonal, heptagonal, or octagonal (e.g., having a cross-section of a shape as described herein).

The indexed tube array housing (7) may include a plurality of tubes (8). For example, the tube array housing may include from 1 to 1,000 tubes, e.g., from 1 to 10 (e.g., 1, 2, 3, 4, 5, 6, 7, 8, 9, or 10), 10 to 100 (e.g., 20, 30, 40, 50, 60, 70, 80, 90, or 100), or 100 to 1,000 (e.g., 200, 300, 400, 500, 600, 700, 800, 900, or 1,000) tubes. In some embodiments, the tube array housing includes 2, 4, 6, 8, 12, 16, 24, 32, 48, 64, 96, 128, 256, or 384 tubes. In some embodiments, the tube array housing (7) includes 12 tubes (8).

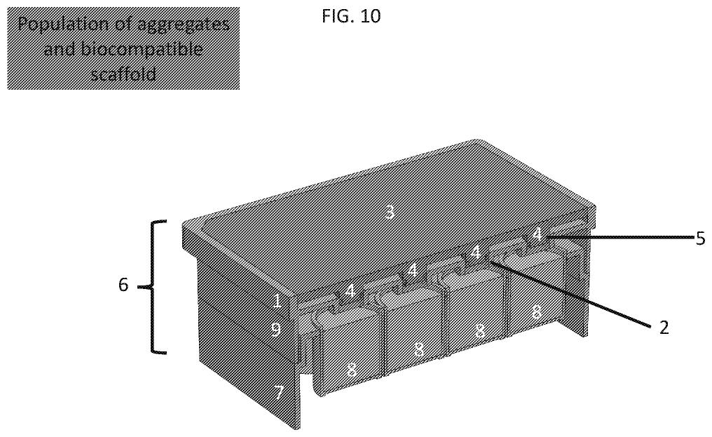

The tube array housing and the well plate may have the same corresponding number of tubes and wells, respectively (see, e.g., FIG. 10).

### Lid (10)

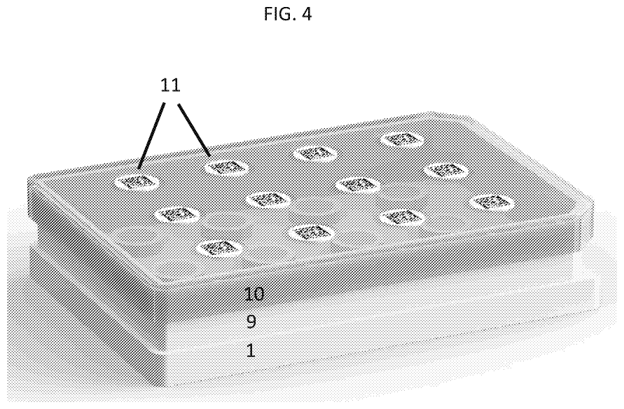

The devices described herein may further include a lid (10). The lid (10) may be removably connected to the well plate (1) to serve as cover for the well plate (1), e.g., during incubation (see, e.g., FIG. 4). The lid may attach to the well plate (1) or the tray (9).

The lid may have a height of, e.g., from 1 mm to 100 mm. For example, in some embodiments, the lid (10) may have a height of from 1 mm to 10 mm (e.g., 1 mm, 2 mm, 3 mm, 4 mm, 5 mm, 6 mm, 7 mm, 8 mm, 9 mm, or 10 mm) or 10 mm to 100 mm (e.g., 20 mm, 30 mm, 40 mm, 50 mm, 60 mm, 70 mm, 80 mm, 90 mm, or 100 mm).

In some embodiments, the length and/or width of the lid (10) is, independently from 1 cm to about 100 cm. For example, the length and/or width of the lid (10) may be, independently 1 cm to 10 cm (e.g., 1 cm, 2 cm, 3 cm, 4 cm, 5 cm, 6 cm, 7 cm, 8 cm, 9 cm, or 10 cm), 10 cm to 20 cm (e.g., 11 cm, 12 cm, 13 cm, 14 cm, 15 cm, 16 cm, 17 cm, 18 cm, 19 cm, or 20 cm), 20 cm to 50 cm (e.g., 25 cm, 30 cm, 35 cm, 40 cm, 45 cm, or 50 cm), or 50 cm to 100 cm (e.g., 60 cm, 70 cm, 80 cm, 90 cm, or 100 cm). The lid (10) may be, e.g., a square or rectangular in shape. The lid may have substantially the same length and width as the well plate (1).

In some embodiments, the lid (10) contains one or more sample identification elements (11), such as barcodes (see, e.g., FIG. 4). The sample identification elements may be used to track properties or lot numbers of the aggregates or grafts in a particular well (2).

In some embodiments, the sample identification elements (11) may be present on a different part of the device, such as on well plate (1), wells (2), indexed mold floor (3), or on protrusions (4). The sample identification elements may be present in any location suitable to identify a specific sample.

### Methods of Encapsulation

The invention features a method of using a device as described herein. The device may be used to encapsulate cells or aggregates thereof in a scaffold (e.g., a biocompatible scaffold). Such scaffolds of encapsulated cells may be used to form a graft (e.g., tissue graft) for various downstream uses, such as implantation into a subject (e.g., a human subject). The methods include providing a device as described herein and introducing a population of aggregates and a biocompatible scaffold into the one or more wells (2) of the well plate (1) (see, e.g., FIGS. 6 and 7). The method may further include introducing a polymerizing agent into the one or more wells (2) to polymerize the biocompatible scaffold, thereby encapsulating the population of aggregates, e.g., in the biocompatible scaffold. In other examples, the method may include incubating the device for a time and under conditions sufficient to result in polymerization of the biocompatible scaffold without adding a polymerizing agent. The aggregates may include, for example, two or more populations of cells (e.g., two, three, four, five, six, seven, eight, nine, ten, or more populations of cells). The aggregates may include, for example, a first population of cells and a second population of cells. In some embodiments, the aggregates include a population of stromal cells and a population of parenchymal cells.

The introduction of aggregates of the first population of cells and the second population of cells can be sequential, e.g., the aggregates of the first population of cells may be first introduced, e.g., in a first medium, and the aggregates of the second population of cells may be subsequently introduced, e.g., in a second medium. Similarly, the aggregates of second population cells may be first introduced, e.g., in a first medium, and the aggregates of the first population cells may be subsequently introduced, e.g., in a first medium. Alternatively, the aggregates of the first population of cells and the aggregates of second population of cells may be introduced in the same medium.

The introduction of aggregates of stromal cells and parenchymal cells can be sequential, e.g., the aggregates of stromal cells may be first introduced, e.g., in a first medium, and the aggregates of parenchymal cells may be subsequently introduced, e.g., in a second medium. Similarly, the aggregates of parenchymal cells may be first introduced, e.g., in a first medium, and the aggregates of stromal cells may be subsequently introduced, e.g., in a second medium. Alternatively, the aggregates of stromal cells and the aggregates of parenchymal cells may be introduced in the same medium. In some embodiments, the aggregates include a combination of stromal cells and parenchymal cells, which are introduced in a single medium. In some embodiments, the method includes adding aggregates of stromal cells and parenchymal cells in multiple additions. For example, a first population may be introduced into the wells (2) followed by a second population of aggregates being introduced into the wells (2).

In some embodiments, the biocompatible scaffold includes fibrin. In some embodiments, the polymerizing agent includes thrombin. Following introduction of a polymerizing agent (e.g., thrombin), the fibrinogen in the scaffold is polymerized into fibrin.

In some embodiments, the biocompatible scaffold further includes a reinforcing agent. For example, the reinforcing agent may include collagen, poly(ethylene glycol), polyvinylidene acetate (PVDA), polyvinylidene fluoride (PVDF), poly(lactic-co-glycolic) acid (PLGA), or poly (l-lactic acid) (PLLA).

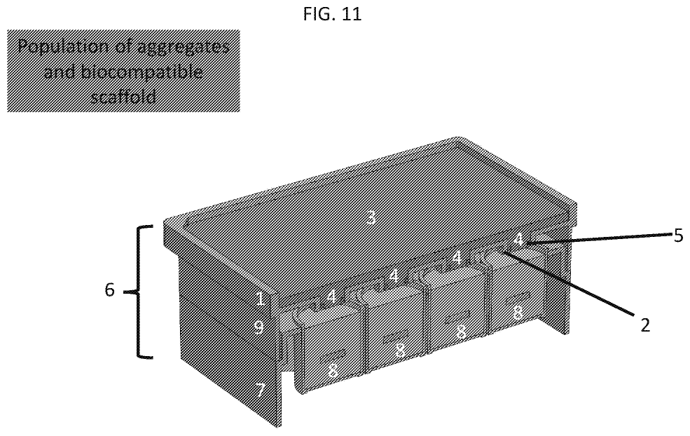

Also contemplated by the methods described herein is a method of releasing an encapsulated population of aggregates from the wells. The method may include raising the indexed mold floor (3) to eject the encapsulated population of aggregates from the one or more wells (2) (see, e.g., FIG. 3). The protrusions (4) may vertically insert into the wells (2) to eject the grafts. The encapsulated population of aggregates may be released, e.g., into a vessel (6) or a storage tube (8) therein (see, e.g., FIG. 11).

In some embodiments, the method further includes distributing the encapsulated population of aggregates from the one or more wells (2) into the one or more storage tubes (8). In some embodiments, the encapsulated population of aggregates is distributed by gravity.

In some embodiments, the parenchymal cells are hepatocytes, pancreatic exocrine cells, myocytes, pancreatic endocrine cells, neurons, enterocytes, adipocytes, splenic cells, kidney cells, biliary cells, Kupffer cells, stellate cells, cardiac muscle cells, alveolar cells, bronchiolar cells, club cells, urothelial cells, mucous cells, parietal cells, chief cells, G cells, goblet cells, enteroendocrine cells, Paneth cells, M cells, tuft cells, glial cells, gall bladder cells, keratinocytes, melanocytes, Merkel cells, Langerhans cells, osteocytes, osteoclasts, esophageal cells, photoreceptor cells, or corneal epithelial cells. In some embodiments, the parenchymal cells are pancreatic cells (e.g., alpha, beta, gamma, delta, epsilon cells, or any combination thereof). In some embodiments, the parenchymal cells comprise beta cells.

In some embodiments, the hepatocytes are primary human hepatocytes.

In some embodiments, the stromal cells are fibroblasts, endothelial cells, or pericytes.

In some embodiments, the fibroblasts are normal human dermal fibroblasts or neonatal foreskin fibroblasts. In some embodiments, the fibroblasts are normal human dermal fibroblasts.

The invention features a method of encapsulating a population of aggregates. Such aggregates may be formed by any suitable method known in the art. Methods of aggregation may include aggregating a population of cells, such as a population of stromal cells and/or a population of parenchymal cells.

Also contemplated herein is an encapsulated population of aggregates of a first population of cells (e.g., stromal cells) and a second population of cells (e.g., parenchymal cells) produced by a method as described herein. In some embodiments, the encapsulated population of aggregates is suitable for implantation into a subject (e.g., a human subject).

### Cell Populations

Cell populations may be optimized to maintain the appropriate morphology, phenotype, and cellular function conducive to use in the methods and devices of the disclosure. For example, primary human hepatocytes or neonatal foreskin stromal cells can be isolated and/or pre-cultured under conditions optimized to ensure that the respective cells of choice initially have the desired morphology, phenotype, and cellular function and, thus, are poised to maintain said morphology, phenotype and/or function while being encapsulated in a device described herein.

In some embodiments, a method described herein may include providing a first population of cells and a second population of cells.

In some embodiments, a method described herein may include providing a population of parenchymal cells (e.g., hepatocytes, pancreatic exocrine cells, myocytes, pancreatic endocrine cells, neurons, enterocytes, adipocytes, splenic cells, kidney cells, biliary cells, Kupffer cells, stellate cells, cardiac muscle cells, alveolar cells, bronchiolar cells, club cells, urothelial cells, mucous cells, parietal cells, chief cells, G cells, goblet cells, enteroendocrine cells, Paneth cells, M cells, tuft cells, glial cells, gall bladder cells, keratinocytes, melanocytes, Merkel cells, Langerhans cells, osteocytes, osteoclasts, esophageal cells, photoreceptor cells, and corneal epithelial cells). In some embodiments, the parenchymal cells are pancreatic cells (e.g., alpha, beta, gamma, delta, epsilon cells, or any combination thereof).

In some embodiments the first population of cells and/or the second population of cells are induced pluripotent (iPSC)-derived cells, engineered cells, primary cells, embryonic stem cells (ESC)-derived cells, or transdifferentiated cells. In some embodiments, the primary cells include primary cells expanded in vitro.

In some embodiments, the engineered cells are engineered to express or secrete a protein (e.g., an antibody, a cytokine, an enzyme, a coagulation factor, or a hormone). In some embodiments, the protein is an endogenous human protein or an engineered protein.

In some embodiments, the first and/or second population of cells includes endocrine, exocrine, paracrine, heterocrine, autocrine, or juxtacrine cells.

In some embodiments, the first and/or second population of cells includes leading cells, adrenal cortical cells, pituitary cells, thyrocytes, granulosa cells, mammary gland epithelial cells, thymocytes, thymic epithelial cells, hypothalamus cells, skeletal muscle cells, smooth muscle cells, and/or neuronal cells.

In some embodiments, the pituitary cells include thyrotropic pituitary cells, lactotropic pituitary cells, corticotropic pituitary cells, somatotropic pituitary cells, and/or gonadotropic pituitary cells. In some embodiments, the neuronal cells include dopaminergic cells.

In some embodiments, the first and/or second population of cells includes parenchymal cells (e.g., hepatocytes, pancreatic exocrine cells, myocytes, pancreatic endocrine cells, neurons, enterocytes, adipocytes, splenic cells, kidney cells, biliary cells, Kupffer cells, stellate cells, cardiac muscle cells, alveolar cells, bronchiolar cells, club cells, urothelial cells, mucous cells, parietal cells, chief cells, G cells, goblet cells, enteroendocrine cells, Paneth cells, M cells, tuft cells, glial cells, gall bladder cells, keratinocytes, melanocytes, Merkel cells, Langerhans cells, osteocytes, osteoclasts, esophageal cells, photoreceptor cells, and corneal epithelial cells). In some embodiments, the parenchymal cells are pancreatic cells (e.g., alpha, beta, gamma, delta, epsilon cells, or any combination thereof). In some embodiments, the parenchymal cells include beta cells.

In some embodiments, the first and/or second population of cells are engineered cells, primary cells, or transdifferentiated cells.

In some embodiments, the method includes encapsulating two or more populations of cells (e.g., two, three, four, five, six, seven, eight, nine, ten, or more populations of cells).

In some embodiments, a method described herein may include providing a population of stromal cells.

In some embodiments, the ratio of parenchymal cells to stromal cells is from 1:10 to 4:1 (e.g., 1:10 to 4:1, 1:10 to 3:1, 1:10 to 2:1, 1:10 to 1:1, 1:9 to 4:1, 1:9 to 3:1, 1:9 to 2:1, 1:9 to 1:1, 1:8 to 4:1, 1:8 to 3:1, 1:8 to 2:1, 1:8 to 1:1, 1:7 to 4:1, 1:7 to 3:1, 1:7 to 2:1, 1:7 to 1:1, 1:6 to 4:1, 1:6 to 3:1, 1:6 to 2:1, 1:6 to 1:1, 1:5 to 4:1, 1:5 to 3:1, 1:5 to 2:1, 1:5 to 1:1, 1:4 to 4:1, 1:4 to 3:1, 1:4 to 2:1, 1:4 to 1:1, 1:3 to 4:1, 1:3 to 3:1, 1:3 to 2:1, 1:3 to 1:1, 1:2 to 4:1, 1:2 to 3:1, 1:2 to 2:1, 1:2 to 1:1, 1:1 to 4:1, 1:1 to 3:1, 1:1 to 2:1, or 1:0 to 1:1).

For example, in some embodiments, the ratio of parenchymal cells to stromal cells is from 1:9 to 4:1. In some embodiments, the ratio of parenchymal cells to stromal cells is from 1:8 to 4:1. In some embodiments, the ratio of parenchymal cells to stromal cells is from 1:7 to 4:1. In some embodiments, the ratio of parenchymal cells to stromal cells is from 1:6 to 4:1. In some embodiments, the ratio of parenchymal cells to stromal cells is from 1:5 to 4:1. In some embodiments, the ratio of parenchymal cells to stromal cells is from 1:4 to 4:1. In some embodiments, the ratio of parenchymal cells to stromal cells is from 1:3 to 4:1. In some embodiments, the ratio of parenchymal cells to stromal cells is from 1:2 to 4:1. In some embodiments, the ratio of parenchymal cells to stromal cells is from 1:1 to 4:1. In some embodiments, the ratio of parenchymal cells to stromal cells is from 1:0 to 4:1.

In some embodiments, the ratio of parenchymal cells to stromal cells is from 1:10 to 3:1. In some embodiments, the ratio of parenchymal cells to stromal cells is from 1:9 to 3:1. In some embodiments, the ratio of parenchymal cells to stromal cells is from 1:8 to 3:1. In some embodiments, the ratio of parenchymal cells to stromal cells is from 1:7 to 3:1. In some embodiments, the ratio of parenchymal cells to stromal cells is from 1:6 to 3:1. In some embodiments, the ratio of parenchymal cells to stromal cells is from 1:5 to 3:1. In some embodiments, the ratio of parenchymal cells to stromal cells is from 1:4 to 3:1. In some embodiments, the ratio of parenchymal cells to stromal cells is from 1:3 to 3:1. In some embodiments, the ratio of parenchymal cells to stromal cells is from 1:2 to 3:1. In some embodiments, the ratio of parenchymal cells to stromal cells is from 1:1 to 3:1. In some embodiments, the ratio of parenchymal cells to stromal cells is from 1:0 to 3:1.

In some embodiments, the ratio of parenchymal cells to stromal cells is from 1:10 to 2:1. In some embodiments, the ratio of parenchymal cells to stromal cells is from 1:9 to 2:1. In some embodiments, the ratio of parenchymal cells to stromal cells is from 1:8 to 2:1. In some embodiments, the ratio of parenchymal cells to stromal cells is from 1:7 to 2:1. In some embodiments, the ratio of parenchymal cells to stromal cells is from 1:6 to 2:1. In some embodiments, the ratio of parenchymal cells to stromal cells is from 1:5 to 2:1. In some embodiments, the ratio of parenchymal cells to stromal cells is from 1:4 to 2:1. In some embodiments, the ratio of parenchymal cells to stromal cells is from 1:3 to 2:1. In some embodiments, the ratio of parenchymal cells to stromal cells is from 1:2 to 2:1. In some embodiments, the ratio of parenchymal cells to stromal cells is from 1:1 to 2:1. In some embodiments, the ratio of parenchymal cells to stromal cells is from 1:0 to 2:1.

In some embodiments, the ratio of parenchymal cells to stromal cells is from 1:10 to 1:1. In some embodiments, the ratio of parenchymal cells to stromal cells is from 1:9 to 1:1. In some embodiments, the ratio of parenchymal cells to stromal cells is from 1:8 to 1:1. In some embodiments, the ratio of parenchymal cells to stromal cells is from 1:7 to 1:1. In some embodiments, the ratio of parenchymal cells to stromal cells is from 1:6 to 1:1. In some embodiments, the ratio of parenchymal cells to stromal cells is from 1:5 to 1:1. In some embodiments, the ratio of parenchymal cells to stromal cells is from 1:4 to 1:1. In some embodiments, the ratio of parenchymal cells to stromal cells is from 1:3 to 1:1. In some embodiments, the ratio of parenchymal cells to stromal cells is from 1:2 to 1:1. In some embodiments, the ratio of parenchymal cells to stromal cells is from 1:1 to 1:0.

Using any of the methods described herein, the disclosure provides a composition including a population of aggregates that includes stromal cells and parenchymal cells produced by said methods.

### Parenchymal Cells

A device described herein can be used to encapsulate one or more populations of cells or aggregates thereof, one of which may include parenchymal cells (e.g., hepatocytes, pancreatic exocrine cells, myocytes, pancreatic endocrine cells, neurons, enterocytes, adipocytes, splenic cells, kidney cells, biliary cells, Kupffer cells, stellate cells, cardiac muscle cells, alveolar cells, bronchiolar cells, club cells, urothelial cells, mucous cells, parietal cells, chief cells, G cells, goblet cells, enteroendocrine cells, Paneth cells, M cells, tuft cells, glial cells, gall bladder cells, keratinocytes, melanocytes, Merkel cells, Langerhans cells, osteocytes, osteoclasts, esophageal cells, photoreceptor cells, and corneal epithelial cells). For example, in some embodiments, the parenchymal cells are hepatocytes. In some embodiments, the parenchymal cells are pancreatic exocrine cells. In some embodiments, the parenchymal cells are myocytes. In some embodiments, the parenchymal cells are pancreatic endocrine cells. In some embodiments, the parenchymal cells are neurons. In some embodiments, the parenchymal cells are enterocytes. In some embodiments, the parenchymal cells are adipocytes. In some embodiments, the parenchymal cells are splenic cells. In some embodiments, the parenchymal cells are kidney cells. In some embodiments, the parenchymal cells are biliary cells. In some embodiments, the parenchymal cells are Kupffer cells. In some embodiments, the parenchymal cells are stellate cells. In some embodiments, the parenchymal cells are cardiac muscle cells. In some embodiments, the parenchymal cells are alveolar cells. In some embodiments, the parenchymal cells are bronchiolar cells. In some embodiments, the parenchymal cells are club cells. In some embodiments, the parenchymal cells are urothelial cells. In some embodiments, the parenchymal cells are mucous cells. In some embodiments, the parenchymal cells are parietal cells. In some embodiments, the parenchymal cells are chief cells. In some embodiments, the parenchymal cells are G cells. In some embodiments, the parenchymal cells are goblet cells. In some embodiments, the parenchymal cells are enteroendocrine cells. In some embodiments, the parenchymal cells are Paneth cells. In some embodiments, the parenchymal cells are M cells. In some embodiments, the parenchymal cells are tuft cells. In some embodiments, the parenchymal cells are glial cells. In some embodiments, the parenchymal cells are gall bladder cells. In some embodiments, the parenchymal cells are keratinocytes. In some embodiments, the parenchymal cells are melanocytes. In some embodiments, the parenchymal cells are Merkel cells. In some embodiments, the parenchymal cells are Langerhans cells. In some embodiments, the parenchymal cells are osteocytes. In some embodiments, the parenchymal cells are osteoclasts. In some embodiments, the parenchymal cells are esophageal cells. In some embodiments, the parenchymal cells are photoreceptor cells. In some embodiments, the parenchymal cells are corneal epithelial cells. In some embodiments, the parenchymal cells are pancreatic cells (e.g., alpha, beta, gamma, delta, epsilon cells, or any combination thereof). In some embodiments, the parenchymal cells are alpha cells. In some embodiments, the parenchymal cells are beta cells. In some embodiments, the parenchymal cells are gamma cells. In some embodiments, the parenchymal cells are delta cells. In some embodiments, the parenchymal cells are epsilon cells.

In some embodiments, the parenchymal cells are hepatocytes, e.g., primary human hepatocytes (PHH).

In some embodiments, the ratio of hepatocytes to stromal cells is from 1:10 to 4:1 (e.g., 1:10 to 4:1, 1:10 to 3:1, 1:10 to 2:1, 1:10 to 1:1, 1:9 to 4:1, 1:9 to 3:1, 1:9 to 2:1, 1:9 to 1:1, 1:8 to 4:1, 1:8 to 3:1, 1:8 to 2:1, 1:8 to 1:1, 1:7 to 4:1, 1:7 to 3:1, 1:7 to 2:1, 1:7 to 1:1, 1:6 to 4:1, 1:6 to 3:1, 1:6 to 2:1, 1:6 to 1:1, 1:5 to 4:1, 1:5 to 3:1, 1:5 to 2:1, 1:5 to 1:1, 1:4 to 4:1, 1:4 to 3:1, 1:4 to 2:1, 1:4 to 1:1, 1:3 to 4:1, 1:3 to 3:1, 1:3 to 2:1, 1:3 to 1:1, 1:2 to 4:1, 1:2 to 3:1, 1:2 to 2:1, 1:2 to 1:1, 1:1 to 4:1, 1:1 to 3:1, 1:1 to 2:1, or 1:0 to 1:1).

For example, in some embodiments, the ratio of hepatocytes to stromal cells is from 1:9 to 4:1. In some embodiments, the ratio of hepatocytes to stromal cells is from 1:8 to 4:1. In some embodiments, the ratio of hepatocytes to stromal cells is from 1:7 to 4:1. In some embodiments, the ratio of hepatocytes to stromal cells is from 1:6 to 4:1. In some embodiments, the ratio of hepatocytes to stromal cells is from 1:5 to 4:1. In some embodiments, the ratio of hepatocytes to stromal cells is from 1:4 to 4:1. In some embodiments, the ratio of hepatocytes to stromal cells is from 1:3 to 4:1. In some embodiments, the ratio of hepatocytes to stromal cells is from 1:2 to 4:1. In some embodiments, the ratio of hepatocytes to stromal cells is from 1:1 to 4:1. In some embodiments, the ratio of hepatocytes to stromal cells is from 1:0 to 4:1.

In some embodiments, the ratio of hepatocytes to stromal cells is from 1:10 to 3:1. In some embodiments, the ratio of hepatocytes to stromal cells is from 1:9 to 3:1. In some embodiments, the ratio of hepatocytes to stromal cells is from 1:8 to 3:1. In some embodiments, the ratio of hepatocytes to stromal cells is from 1:7 to 3:1. In some embodiments, the ratio of hepatocytes to stromal cells is from 1:6 to 3:1. In some embodiments, the ratio of hepatocytes to stromal cells is from 1:5 to 3:1. In some embodiments, the ratio of hepatocytes to stromal cells is from 1:4 to 3:1. In some embodiments, the ratio of hepatocytes to stromal cells is from 1:3 to 3:1. In some embodiments, the ratio of hepatocytes to stromal cells is from 1:2 to 3:1. In some embodiments, the ratio of hepatocytes to stromal cells is from 1:1 to 3:1. In some embodiments, the ratio of hepatocytes to stromal cells is from 1:0 to 3:1.

In some embodiments, the ratio of hepatocytes to stromal cells is from 1:10 to 2:1. In some embodiments, the ratio of hepatocytes to stromal cells is from 1:9 to 2:1. In some embodiments, the ratio of hepatocytes to stromal cells is from 1:8 to 2:1. In some embodiments, the ratio of hepatocytes to stromal cells is from 1:7 to 2:1. In some embodiments, the ratio of hepatocytes to stromal cells is from 1:6 to 2:1. In some embodiments, the ratio of hepatocytes to stromal cells is from 1:5 to 2:1. In some embodiments, the ratio of hepatocytes to stromal cells is from 1:4 to 2:1. In some embodiments, the ratio of hepatocytes to stromal cells is from 1:3 to 2:1. In some embodiments, the ratio of hepatocytes to stromal cells is from 1:2 to 2:1. In some embodiments, the ratio of hepatocytes to stromal cells is from 1:1 to 2:1. In some embodiments, the ratio of hepatocytes to stromal cells is from 1:0 to 2:1.

In some embodiments, the ratio of hepatocytes to stromal cells is from 1:10 to 1:1. In some embodiments, the ratio of hepatocytes to stromal cells is from 1:9 to 1:1. In some embodiments, the ratio of hepatocytes to stromal cells is from 1:8 to 1:1. In some embodiments, the ratio of hepatocytes to stromal cells is from 1:7 to 1:1. In some embodiments, the ratio of hepatocytes to stromal cells is from 1:6 to 1:1. In some embodiments, the ratio of hepatocytes to stromal cells is from 1:5 to 1:1. In some embodiments, the ratio of hepatocytes to stromal cells is from 1:4 to 1:1. In some embodiments, the ratio of hepatocytes to stromal cells is from 1:3 to 1:1. In some embodiments, the ratio of hepatocytes to stromal cells is from 1:2 to 1:1. In some embodiments, the ratio of hepatocytes to stromal cells is from 1:1 to 1:0.

In some embodiments, the parenchymal cells are PHH.

In some embodiments, the population of PHH includes an amount of from 2.5×10⁴ to 1.8×10¹¹ (e.g., from 3×10⁴ to 1.8×10¹¹, 4×10⁴ to 1.8×10¹¹, 5×10⁴ to 1.8×10¹¹, 1×10⁵ to 1.8×10¹¹, 4×10⁵ to 1.8×10¹¹, 5×10⁵ to 1.8×10¹¹, 6×10⁵ to 1.8×10¹¹, 7×10⁵ to 1.8×10¹¹, 8×10⁵ to 1.8×10¹¹, 9×10⁵ to 1.8×10¹¹, 1×10⁶ to 1.8×10¹¹, 2×10⁶ to 1.8×10¹¹, 3×10⁶ to 1.8×10¹¹, 4×10⁶ to 1.8×10¹¹, 5×10⁶ to 1.8×10¹¹, 6×10⁶ to 1.8×10¹¹, 7×10⁶ to 1.8×10¹¹, 8×10⁶ to 1.8×10¹¹, 9×10⁶ to 1.8×10¹¹, 1×10⁷ to 1.8×10¹¹, 2×10⁷ to 1.8×10¹¹, 1.8×10⁷ to 1.8×10¹¹, 4×10⁷ to 1.8×10¹¹, 5×10⁷ to 1.8×10¹¹, 6×10⁷ to 1.8×10¹¹, 7×10⁷ to 1.8×10¹¹, 8×10⁷ to 1.8×10¹¹, 9×10⁷ to 1.8×10¹¹, 1×10⁸ to 1.8×10¹¹, 2×10⁸ to 1.8×10¹¹, 3×10⁸ to 1.8×10¹¹, 4×10⁸ to 1.8×10¹¹, 5×10⁸ to 1.8×10¹¹, 6×10⁸ to 1.8×10¹¹, 7×10⁸ to 1.8×10¹¹, 8×10⁸ to 1.8×10¹¹, 9×10⁸ to 1.8×10¹¹, 1×10⁹ to 1.8×10¹¹, 2×10⁹ to 1.8×10¹¹, 3×10⁹ to 1.8×10¹¹, 4×10⁹ to 1.8×10¹¹, 5×10⁹ to 1.8×10¹¹, 6×10⁹ to 1.8×10¹¹, 7×10⁹ to 1.8×10¹¹, 8×10⁹ to 1.8×10¹¹, 9×10⁹ to 1.8×10¹¹, 1×10¹⁰ to 1.8×10¹¹, 2×10¹⁰ to 1.8×10¹¹, 3×10¹⁰ to 1.8×10¹¹, 4×10¹⁰ to 1.8×10¹¹, 5×10¹⁰ to 1.8×10¹¹, 6×10¹⁰ to 1.8×10¹¹, 7×10¹⁰ to 1.8×10¹¹, 8×10¹⁰ to 1.8×10¹¹, 9×10¹⁰ to 1.8×10¹¹, or 1×10¹¹ to 1.8×10¹¹) PHH. For example, in some embodiments, the population of PHH includes an amount of from 3×10⁴ to 1.8×10¹¹. In some embodiments, the population of PHH includes an amount of from 4×10⁴ to 1.8×10¹¹. In some embodiments, the population of PHH includes an amount of from 5×10⁴ to 1.8×10¹¹. In some embodiments, the population of PHH includes an amount of from 1×10⁵ to 1.8×10¹¹. In some embodiments, the population of PHH includes an amount of from 2×10⁵ to 1.8×10¹¹. In some embodiments, the population of PHH includes an amount of from 3×10⁵ to 1.8×10¹¹ PHH. In some embodiments, the population of PHH includes an amount of from 4×10⁵ to 1.8×10¹¹ PHH. In some embodiments, the population of PHH includes an amount of from 5×10⁵ to 1.8×10¹¹ PHH. In some embodiments, the population of PHH includes an amount of from 6×10⁵ to 1.8×10¹¹ PHH. In some embodiments, the population of PHH includes an amount of from 7×10⁵ to 1.8×10¹¹ PHH. In some embodiments, the population of PHH includes an amount of from 8×10⁵ to 1.8×10¹¹ PHH. In some embodiments, the population of PHH includes an amount of from 9×10⁵ to 1.8×10¹¹ PHH. In some embodiments, the population of PHH includes an amount of from 1×10⁶ to 1.8×10¹¹ PHH. In some embodiments, the population of PHH includes an amount of from 2×10⁶ to 1.8×10¹¹ PHH. In some embodiments, the population of PHH includes an amount of from 3×10⁶ to 1.8×10¹¹ PHH. In some embodiments, the population of PHH includes an amount of from 4×10⁶ to 1.8×10¹¹ PHH. In some embodiments, the population of PHH includes an amount of from 5×10⁶ to 1.8×10¹¹ PHH. In some embodiments, the population of PHH includes an amount of from 6×10⁶ to 1.8×10¹¹ PHH. In some embodiments, the population of PHH includes an amount of from 7×10⁶ to 1.8×10¹¹ PHH. In some embodiments, the population of PHH includes an amount of from 8×10⁶ to 1.8×10¹¹ PHH. In some embodiments, the population of PHH includes an amount of from 9×10⁶ to 1.8×10¹¹. In some embodiments, the population of PHH includes an amount of from 1×10⁷ to 1.8×10¹¹ PHH. In some embodiments, the population of PHH includes an amount of from 2×10⁷ to 1.8×10¹¹ PHH. In some embodiments, the population of PHH includes an amount of from 3×10⁷ to 1.8×10¹¹ PHH. In some embodiments, the population of PHH includes an amount of from 4×10⁷ to 1.8×10¹¹ PHH. In some embodiments, the population of PHH includes an amount of from 5×10⁷ to 1.8×10¹¹ PHH. In some embodiments, the population of PHH includes an amount of from 6×10⁷ to 1.8×10¹¹ PHH. In some embodiments, the population of PHH includes an amount of from 7×10⁷ to 1.8×10¹¹ PHH. In some embodiments, the population of PHH includes an amount of from 8×10⁷ to 1.8×10¹¹ PHH. In some embodiments, the population of PHH includes an amount of from 9×10⁷ to 1.8×10¹¹ PHH. In some embodiments, the population of PHH includes an amount of from 1×10⁸ to 1.8×10¹¹ PHH. In some embodiments, the population of PHH includes an amount of from 2×10⁸ to 1.8×10¹¹ PHH. In some embodiments, the population of PHH includes an amount of from 3×10⁸ to 1.8×10¹¹ PHH. In some embodiments, the population of PHH includes an amount of from 4×10⁸ to 1.8×10¹¹ PHH. In some embodiments, the population of PHH includes an amount of from 5×10⁸ to 1.8×10¹¹ PHH. In some embodiments, the population of PHH includes an amount of from 6×10⁸ to 1.8×10¹¹ PHH. In some embodiments, the population of PHH includes an amount of from 7×10⁸ to 1.8×10¹¹ PHH. In some embodiments, the population of PHH includes an amount of from 8×10⁸ to 1.8×10¹¹ PHH. In some embodiments, the population of PHH includes an amount of from 9×10⁸ to 1.8×10¹¹ PHH. In some embodiments, the population of PHH includes an amount of from 1×10⁹ to 1.8×10¹¹ PHH. In some embodiments, the population of PHH includes an amount of from 2×10⁹ to 1.8×10¹¹ PHH. In some embodiments, the population of PHH includes an amount of from 3×10⁹ to 1.8×10¹¹ PHH. In some embodiments, the population of PHH includes an amount of from 4×10⁹ to 1.8×10¹¹ PHH. In some embodiments, the population of PHH includes an amount of from 5×10⁹ to 1.8×10¹¹ PHH. In some embodiments, the population of PHH includes an amount of from 6×10⁹ to 1.8×10¹¹ PHH. In some embodiments, the population of PHH includes an amount of from 7×10⁹ to 1.8×10¹¹ PHH. In some embodiments, the population of PHH includes an amount of from 8×10⁹ to 1.8×10¹¹ PHH. In some embodiments, the population of PHH includes an amount of from 9×10⁹ to 1.8×10¹¹ PHH. In some embodiments, the population of PHH includes an amount of from 1×10¹⁰ to 1.8×10¹¹ PHH. In some embodiments, the population of PHH includes an amount of from 2×10¹⁰ to 1.8×10¹¹ PHH. In some embodiments, the population of PHH includes an amount of from 3×10¹⁰ to 1.8×10¹¹ PHH. In some embodiments, the population of PHH includes an amount of from 4×10¹⁰ to 1.8×10¹¹ PHH. In some embodiments, the population of PHH includes an amount of from 5×10¹⁰ to 1.8×10¹¹ PHH. In some embodiments, the population of PHH includes an amount of from 6×10¹⁰ to 1.8×10¹¹ PHH. In some embodiments, the population of PHH includes an amount of from 7×10¹⁰ to 1.8×10¹¹ PHH. In some embodiments, the population of PHH includes an amount of from 8×10¹⁰ to 1.8×10¹¹ PHH. In some embodiments, the population of PHH includes an amount of from 9×10¹⁰ to 1.8×10¹¹ PHH. In some embodiments, the population of PHH includes an amount of from 1×10¹¹ to 1.8×10¹¹ PHH.

### Stromal Cells

A device described herein can be used to encapsulate one or more populations of cells or aggregates thereof, one of which may include parenchymal cells stromal cells (e.g., fibroblasts, endothelial cells, or pericytes). For example, in some embodiments, the stromal cells are fibroblasts (e.g., normal human dermal fibroblasts or neonatal foreskin fibroblasts). In some embodiments, the stromal cells are endothelial cells. In some embodiments, the stromal cells are pericytes. In some embodiments, the stromal cells are normal human dermal fibroblasts. In some embodiments, the stromal cells are neonatal foreskin fibroblasts.

In some embodiments, the stromal cells are normal human dermal fibroblasts.

In some embodiments, the population of stromal cells (e.g., fibroblasts) is up to 1.8×10¹² (e.g., from 1 to 1.8×10¹², from 10 to 1.8×10¹², from 100 to 1.8×10¹², from 1×10³ to 1.8×10¹², from 2×103to 1.8×10¹², from 3×10³ to 1.8×10¹², from 4×10³ to 1.8×10¹², from 5×10³ to 1.8×10¹², from 6×103to 1.8×10¹², from 7×10³ to 1.8×10¹², from 8×10³ to 1.8×10¹², from 9×10³ to 1.8×10¹², from 1×104to 1.8×10¹², from 2×10⁴ to 1.8×10¹², from 3×10⁴ to 1.8×10¹², from 4×10⁴ to 1.8×10¹², from 5×104to 1.8×10¹², from 6×10⁴ to 1.8×10¹², from 7×10⁴ to 1.8×10¹², from 8×10⁴ to 1.8×10¹², from 9×104to 1.8×10¹², from 1×10⁵ to 1.8×10¹², from 2×10⁵ to 1.8×10¹², from 3×10⁵ to 1.8×10¹², from 4×105to 1.8×10¹², from 5×10⁵ to 1.8×10¹², from 6×10⁵ to 1.8×10¹², from 7×10⁵ to 1.8×10¹², from 8×105to 1.8×10¹², from 9×10⁵ to 1.8×10¹², from 1×10⁶ to 1.8×10¹², from 2×10⁶ to 1.8×10¹², 3×10⁶ to 1.8×10¹², 4×10⁶ to 1.8×10¹², 5×10⁶ to 1.8×10¹², 6×10⁶ to 1.8×10¹², 7×10⁶ to 1.8×10¹², 8×10⁶ to 1.8×10¹², 9×10⁶ to 1.8×10¹², from 1×10⁷ to 1.8×10¹², from 2×10⁷ to 1.8×10¹², from 3×10⁷ to 1.8×10¹², from 4×10⁷ to 1.8×10¹², from 5×10⁷ to 1.8×10¹², from 6×10⁷ to 1.8×10¹², from 7×10⁷ to 1.8×10¹², from 8×10⁷ to 1.8×10¹², from 9×10⁷ to 1.8×10¹², from 1×10⁸ to 1.8×10¹², from 2×10⁸ to 1.8×10¹², from 3×10⁸ to 1.8×10¹², from 4×10⁸ to 1.8×10¹², from 5×10⁸ to 1.8×10¹², from 6×10⁸ to 1.8×10¹², from 7×10⁸ to 1.8×10¹², from 8×10⁸ to 1.8×10¹², from 9×10⁸ to 1.8×10¹², from 1×10⁹ to 1.8×10¹², from 2×10⁹ to 1.8×10¹², from 3×10⁹ to 1.8×10¹², from 4×10⁹ to 1.8×10¹², from 5×10⁹ to 1.8×10¹², from 6×10⁹ to 1.8×10¹², from 7×10⁹ to 1.8×10¹², from 8×10⁹ to 1.8×10¹², from 9×10⁹ to 1.8×10¹², from 1×10¹⁰ to 1.8×10¹², from 2×10¹⁰ to 1.8×10¹², from 3×10¹⁰ to 1.8×10¹², from 4×10¹⁰ to 1.8×10¹², from 5×10¹⁰ to 1.8×10¹², from 6×10¹⁰ to 1.8×10¹², from 7×10¹⁰ to 1.8×10¹², from 8×10¹⁰ to 1.8×10¹², from 9×10¹⁰ to 1.8×10¹², from 1×10¹¹ to 1.8×10¹², from 2×10¹¹ to 1.8×10¹², from 3×10¹¹ to 1.8×10¹², from 4×10¹¹ to 1.8×10¹², from 5×10¹¹ to 1.8×10¹², from 6×10¹¹ to 1.8×10¹², from 7×10¹¹ to 1.8×10¹², from 8×10¹¹ to 1.8×10¹², from 9×10¹¹ to 1.8×10¹², or from 1×10¹² to 1.8×10¹²) stromal cells. For example, in some embodiments, the population of stromal cells includes an amount of from 1 to 1.8×10¹² stromal cells. In some embodiments, the population of stromal cells includes an amount of from 10 to 1.8×10¹² stromal cells. In some embodiments, the population of stromal cells includes an amount of from 100 to 1.8×10¹² stromal cells. In some embodiments, the population of stromal cells includes an amount of from 1×10³ to 1.8×10¹² stromal cells. In some embodiments, the population of stromal cells includes an amount from 2×10³ to 1.8×10¹² stromal cells. In some embodiments, the population of stromal cells includes an amount of from 3×10³ to 1.8×10¹² stromal cells. In some embodiments, the population of stromal cells includes an amount of from 4×10³ to 1.8×10¹² stromal cells. In some embodiments, the population of stromal cells includes an amount of from 5×10³ to 1.8×10¹² stromal cells. In some embodiments, the population of stromal cells includes an amount of from 6×10³ to 1.8×10¹² stromal cells. In some embodiments, the population of stromal cells includes an amount of from 7×10³ to 1.8×10¹² stromal cells. In some embodiments, the population of stromal cells includes an amount of from 8×10³ to 1.8×10¹² stromal cells. In some embodiments, the population of stromal cells includes an amount of from 9×10³ to 1.8×10¹² stromal cells. In some embodiments, the population of stromal cells includes an amount of from 1×10⁴ to 1.8×10¹² stromal cells. In some embodiments, the population of stromal cells includes an amount of from 2×10⁴ to 1.8×10¹² stromal cells. In some embodiments, the population of stromal cells includes an amount of from 3×10⁴ to 1.8×10¹² stromal cells. In some embodiments, the population of stromal cells includes an amount of from 4×10⁴ to 1.8×10¹² stromal cells. In some embodiments, the population of stromal cells includes an amount of from 5×10⁴ to 1.8×10¹² stromal cells. In some embodiments, the population of stromal cells includes an amount of from 6×10⁴ to 1.8×10¹² stromal cells. In some embodiments, the population of stromal cells includes an amount of from 7×10⁴ to 1.8×10¹² stromal cells. In some embodiments, the population of stromal cells includes an amount of from 8×10⁴ to 1.8×10¹² stromal cells. In some embodiments, the population of stromal cells includes an amount of from 9×10⁴ to 1.8×10¹² stromal cells. In some embodiments, the population of stromal cells includes an amount of from 1×10⁵ to 1.8×10¹² stromal cells. In some embodiments, the population of stromal cells includes an amount of from 2×10⁵ to 1.8×10¹² stromal cells. In some embodiments, the population of stromal cells includes an amount of from 3×10⁵ to 1.8×10¹² stromal cells. In some embodiments, the population of stromal cells includes an amount of from 4×10⁵ to 1.8×10¹² stromal cells. In some embodiments, the population of stromal cells includes an amount of from 5×10⁵ to 1.8×10¹² stromal cells. In some embodiments, the population of stromal cells includes an amount of from 6×10⁵ to 1.8×10¹² stromal cells. In some embodiments, the population of stromal cells includes an amount of from 7×10⁵ to 1.8×10¹² stromal cells. In some embodiments, the population of stromal cells includes an amount of from 8×10⁵ to 1.8×10¹² stromal cells. In some embodiments, the population of stromal cells includes an amount of from 9×10⁵ to 1.8×10¹² stromal cells. In some embodiments, the population of stromal cells includes an amount of from 1×10⁶ to 1.8×10¹² stromal cells. In some embodiments, the population of stromal cells includes an amount of from 2×10⁶ to 1.8×10¹² stromal cells. In some embodiments, the population of stromal cells includes an amount of from 3×10⁶ to 1.8×10¹² stromal cells. In some embodiments, the population of stromal cells includes an amount of from 4×10⁶ to 1.8×10¹² stromal cells. In some embodiments, the population of stromal cells includes an amount of from 5×10⁶ to 1.8×10¹² stromal cells. In some embodiments, the population of stromal cells includes an amount of from 6×10⁶ to 1.8×10¹² stromal cells. In some embodiments, the population of stromal cells includes an amount of from 7×10⁶ to 1.8×10¹² stromal cells. In some embodiments, the population of stromal cells includes an amount of from 8×10⁶ to 1.8×10¹² stromal cells. In some embodiments, the population of stromal cells includes an amount of from 9×10⁶ to 1.8×10¹² stromal cells. In some embodiments, the population of stromal cells includes an amount of from 1×10⁷ to 1.8×10¹² stromal cells. In some embodiments, the population of stromal cells includes an amount of from 2×10⁷ to 1.8×10¹² stromal cells. In some embodiments, the population of stromal cells includes an amount of from 3×10⁷ to 1.8×10¹² stromal cells. In some embodiments, the population of stromal cells includes an amount of from 4×10⁷ to 1.8×10¹² stromal cells. In some embodiments, the population of stromal cells includes an amount of from 5×10⁷ to 1.8×10¹² stromal cells. In some embodiments, the population of stromal cells includes an amount of from 6×10⁷ to 1.8×10¹² stromal cells. In some embodiments, the population of stromal cells includes an amount of from 7×10⁷ to 1.8×10¹² stromal cells. In some embodiments, the population of stromal cells includes an amount of from 8×10⁷ to 1.8×10¹² stromal cells. In some embodiments, the population of stromal cells includes an amount of from 9×10⁷ to 1.8×10¹² stromal cells. In some embodiments, the population of stromal cells includes an amount of from 1×10⁸ to 1.8×10¹² stromal cells. In some embodiments, the population of stromal cells includes an amount of from 2×10⁸ to 1.8×10¹² stromal cells. In some embodiments, the population of stromal cells includes an amount of from 3×10⁸ to 1.8×10¹² stromal cells. In some embodiments, the population of stromal cells includes an amount of from 4×10⁸ to 1.8×10¹² stromal cells. In some embodiments, the population of stromal cells includes an amount of from 5×10⁸ to 1.8×10¹² stromal cells. In some embodiments, the population of stromal cells includes an amount of from 6×10⁸ to 1.8×10¹² stromal cells. In some embodiments, the population of stromal cells includes an amount of from 7×10⁸ to 1.8×10¹² stromal cells. In some embodiments, the population of stromal cells includes an amount of from 8×10⁸ to 1.8×10¹² stromal cells. In some embodiments, the population of stromal cells includes an amount of from 9×10⁸ to 1.8×10¹² stromal cells. In some embodiments, the population of stromal cells includes an amount of from 1×10⁹ to 1.8×10¹² stromal cells. In some embodiments, the population of stromal cells includes an amount of from 2×10⁹ to 1.8×10¹² stromal cells. In some embodiments, the population of stromal cells includes an amount of from 3×10⁹ to 1.8×10¹² stromal cells. In some embodiments, the population of stromal cells includes an amount of from 4×10⁹ to 1.8×10¹² stromal cells. In some embodiments, the population of stromal cells includes an amount of from 5×10⁹ to 1.8×10¹² stromal cells. In some embodiments, the population of stromal cells includes an amount of from 6×10⁹ to 1.8×10¹² stromal cells. In some embodiments, the population of stromal cells includes an amount of from 7×10⁹ to 1.8×10¹² stromal cells. In some embodiments, the population of stromal cells includes an amount of from 8×10⁹ to 1.8×10¹² stromal cells. In some embodiments, the population of stromal cells includes an amount of from 9×10⁹ to 1.8×10¹² stromal cells. In some embodiments, the population of stromal cells includes an amount of from 1×10¹⁰ to 1.8×10¹² stromal cells. In some embodiments, the population of stromal cells includes an amount of from 2×10¹⁰ to 1.8×10¹² stromal cells. In some embodiments, the population of stromal cells includes an amount of from 3×10¹⁰ to 1.8×10¹² stromal cells. In some embodiments, the population of stromal cells includes an amount of from 4×10¹⁰ to 1.8×10¹² stromal cells. In some embodiments, the population of stromal cells includes an amount of from 5×10¹⁰ to 1.8×10¹² stromal cells. In some embodiments, the population of stromal cells includes an amount of from 6×10¹⁰ to 1.8×10¹² stromal cells. In some embodiments, the population of stromal cells includes an amount of from 7×10¹⁰ to 1.8×10¹² stromal cells. In some embodiments, the population of stromal cells includes an amount of from 8×10¹⁰ to 1.8×10¹² stromal cells. In some embodiments, the population of stromal cells includes an amount of from 9×10¹⁰ to 1.8×10¹² stromal cells. In some embodiments, the population of stromal cells includes an amount of from 1×10¹¹ to 1.8×10¹² stromal cells. In some embodiments, the population of stromal cells includes an amount of from 2×10¹¹ to 1.8×10¹² stromal cells. In some embodiments, the population of stromal cells includes an amount of from 3×10¹¹ to 1.8×10¹² stromal cells. In some embodiments, the population of stromal cells includes an amount of from 4×10¹¹ to 1.8×10¹² stromal cells. In some embodiments, the population of stromal cells includes an amount of from 5×10¹¹ to 1.8×10¹² stromal cells. In some embodiments, the population of stromal cells includes an amount of from 6×10¹¹ to 1.8×10¹² stromal cells. In some embodiments, the population of stromal cells includes an amount of from 7×10¹¹ to 1.8×10¹² stromal cells. In some embodiments, the population of stromal cells includes an amount of from 8×10¹¹ to 1.8×10¹² stromal cells. In some embodiments, the population of stromal cells includes an amount of from 9×10¹¹ to 1.8×10¹² stromal cells. In some embodiments, the population of stromal cells includes an amount of from 1×10¹² to 1.8×10¹² stromal cells.

In some embodiments, the engineered tissue construct includes 0 stromal cells. In some embodiments, the population of stromal cells includes an amount of 1 stromal cell (e.g., fibroblast). In some embodiments, the population of stromal cells includes an amount of 10 stromal cells. In some embodiments, the population of stromal cells includes an amount of 100 stromal cells. In some embodiments, the population of stromal cells includes an amount of 1×10³ stromal cells. In some embodiments, the population of stromal cells includes an amount 2×10³ stromal cells. In some embodiments, the population of stromal cells includes an amount of 3×10³ stromal cells. In some embodiments, the population of stromal cells includes an amount of 4×10³ stromal cells. In some embodiments, the population of stromal cells includes an amount of 5×10³ stromal cells. In some embodiments, the population of stromal cells includes an amount of 6×10³ stromal cells. In some embodiments, the population of stromal cells includes an amount of 7×10³ stromal cells. In some embodiments, the population of stromal cells includes an amount of 8×10³ stromal cells. In some embodiments, the population of stromal cells includes an amount of 9×10³ stromal cells. In some embodiments, the population of stromal cells includes an amount of 1×10⁴ stromal cells. In some embodiments, the population of stromal cells includes an amount of 2×10⁴ stromal cells. In some embodiments, the population of stromal cells includes an amount of 3×10⁴ stromal cells. In some embodiments, the population of stromal cells includes an amount of 4×10⁴ stromal cells. In some embodiments, the population of stromal cells includes an amount of 5×10⁴ stromal cells. In some embodiments, the population of stromal cells includes an amount of 6×10⁴ stromal cells. In some embodiments, the population of stromal cells includes an amount of 7×10⁴ stromal cells. In some embodiments, the population of stromal cells includes an amount of 8×10⁴ stromal cells. In some embodiments, the population of stromal cells includes an amount of 9×10⁴ stromal cells. In some embodiments, the population of stromal cells includes an amount of 1×10⁵ stromal cells. In some embodiments, the population of stromal cells includes an amount of 2×10⁵ stromal cells. In some embodiments, the population of stromal cells includes an amount of 3×10⁵ stromal cells. In some embodiments, the population of stromal cells includes an amount of 4×10⁵ stromal cells. In some embodiments, the population of stromal cells includes an amount of 5×10⁵ stromal cells. In some embodiments, the population of stromal cells includes an amount of 6×10⁵ stromal cells. In some embodiments, the population of stromal cells includes an amount of 7×10⁵ stromal cells. In some embodiments, the population of stromal cells includes an amount of 8×10⁵ stromal cells. In some embodiments, the population of stromal cells includes an amount of 9×10⁵ stromal cells. In some embodiments, the population of stromal cells includes an amount of 1×10⁶ stromal cells. In some embodiments, the population of stromal cells includes an amount of 2×10⁶ stromal cells. In some embodiments, the population of stromal cells includes an amount of 3×10⁶ stromal cells. In some embodiments, the population of stromal cells includes an amount of 4×10⁶ stromal cells. In some embodiments, the population of stromal cells includes an amount of 5×10⁶ stromal cells. In some embodiments, the population of stromal cells includes an amount of 6×10⁶ stromal cells. In some embodiments, the population of stromal cells includes an amount of 7×10⁶ stromal cells. In some embodiments, the population of stromal cells includes an amount of 8×10⁶ stromal cells. In some embodiments, the population of stromal cells includes an amount of 9×10⁶ stromal cells. In some embodiments, the population of stromal cells includes an amount of 1×10⁷ stromal cells. In some embodiments, the population of stromal cells includes an amount of 2×10⁷ stromal cells. In some embodiments, the population of stromal cells includes an amount of 3×10⁷ stromal cells. In some embodiments, the population of stromal cells includes an amount of 4×10⁷ stromal cells. In some embodiments, the population of stromal cells includes an amount of 5×10⁷ stromal cells. In some embodiments, the population of stromal cells includes an amount of 6×10⁷ stromal cells. In some embodiments, the population of stromal cells includes an amount of 7×10⁷ stromal cells. In some embodiments, the population of stromal cells includes an amount of 8×10⁷ stromal cells. In some embodiments, the population of stromal cells includes an amount of 9×10⁷ stromal cells. In some embodiments, the population of stromal cells includes an amount of 1×10⁸ stromal cells. In some embodiments, the population of stromal cells includes an amount of 2×10⁸ stromal cells. In some embodiments, the population of stromal cells includes an amount of 3×10⁸ stromal cells. In some embodiments, the population of stromal cells includes an amount of 4×10⁸ stromal cells. In some embodiments, the population of stromal cells includes an amount of 5×10⁸ stromal cells. In some embodiments, the population of stromal cells includes an amount of 6×10⁸ stromal cells. In some embodiments, the population of stromal cells includes an amount of 7×10⁸ stromal cells. In some embodiments, the population of stromal cells includes an amount of 8×10⁸ stromal cells. In some embodiments, the population of stromal cells includes an amount of 9×10⁸ stromal cells. In some embodiments, the population of stromal cells includes an amount of 1×10⁹ stromal cells. In some embodiments, the population of stromal cells includes an amount of 2×10⁹ stromal cells. In some embodiments, the population of stromal cells includes an amount of 3×10⁹ stromal cells. In some embodiments, the population of stromal cells includes an amount of 4×10⁹ stromal cells. In some embodiments, the population of stromal cells includes an amount of 5×10⁹ stromal cells. In some embodiments, the population of stromal cells includes an amount of 6×10⁹ stromal cells. In some embodiments, the population of stromal cells includes an amount of 7×10⁹ stromal cells. In some embodiments, the population of stromal cells includes an amount of 8×10⁹ stromal cells. In some embodiments, the population of stromal cells includes an amount of 9×10⁹ stromal cells. In some embodiments, the population of stromal cells includes an amount of 1×10¹⁰ stromal cells. In some embodiments, the population of stromal cells includes an amount of 2×10¹⁰ stromal cells. In some embodiments, the population of stromal cells includes an amount of 3×10¹⁰ stromal cells. In some embodiments, the population of stromal cells includes an amount of 4×10¹⁰ stromal cells. In some embodiments, the population of stromal cells includes an amount of 5×10¹⁰ stromal cells. In some embodiments, the population of stromal cells includes an amount of 6×10¹⁰ stromal cells. In some embodiments, the population of stromal cells includes an amount of 7×10¹⁰ stromal cells. In some embodiments, the population of stromal cells includes an amount of 8×10¹⁰ stromal cells. In some embodiments, the population of stromal cells includes an amount of 9×10¹⁰ stromal cells. In some embodiments, the population of stromal cells includes an amount of 1×10¹¹ stromal cells. In some embodiments, the population of stromal cells includes an amount of 2×10¹¹ stromal cells. In some embodiments, the population of stromal cells includes an amount of 3×10¹¹ stromal cells. In some embodiments, the population of stromal cells includes an amount of 4×10¹¹ stromal cells. In some embodiments, the population of stromal cells includes an amount of 5×10¹¹ stromal cells. In some embodiments, the population of stromal cells includes an amount of 6×10¹¹ stromal cells. In some embodiments, the population of stromal cells includes an amount of 7×10¹¹ stromal cells. In some embodiments, the population of stromal cells includes an amount of 8×10¹¹ stromal cells. In some embodiments, the population of stromal cells includes an amount of 9×10¹¹ stromal cells. In some embodiments, the population of stromal cells includes an amount of 1×10¹² stromal cells. In some embodiments, the population of stromal cells includes an amount of 1.8×10¹² stromal cells.

### Kits and Systems

The devices described herein may be employed in the form of a kit or system that includes one or more of the components described herein, and optionally, one or more of a population of aggregates that includes a population of stromal cells and a population of parenchymal, a biocompatible scaffold, a polymerizing agent, a reinforcing agent, buffers, media, and the like.

In one embodiment, the invention features a system that includes a well plate (1) that includes one or more wells (2) and an indexed mold floor (3). The indexed mold floor includes one or more protrusions (4) that are configured to insert into the one or more wells and form a bottom surface for the one or more wells. In some embodiments, the system further includes a vessel. The vessel (6) may include a tube array housing (7) with one or more storage tubes (8). The storage tubes maybe aligned with the one or more wells. In some embodiments, the system further includes a lid (10) configured to cover the well plate. In some embodiments, the system further includes one or more sample identification elements (11), such as barcodes. The one or more sample identification elements may be present on the lid (10)

In another embodiment, the invention features a kit that includes a well plate (1) that includes one or more wells (2) and an indexed mold floor (3). The indexed mold floor includes one or more protrusions (4) that are configured to insert into the one or more wells and form a bottom surface for the one or more wells. In some embodiments, the kit further includes a vessel (6). The vessel may include a tube array housing (7) with one or more storage tubes (8). The storage tubes maybe aligned with the one or more wells. In some embodiments, the kit further includes a lid (10) configured to cover the well plate. In some embodiments, the kit further includes one or more sample identification elements (11), such as barcodes. The one or more sample identification elements may be present on the lid (10). In some embodiments, the kit further includes one or more of a population of aggregates that includes a population of stromal cells and a population of parenchymal, a biocompatible scaffold, and a polymerizing agent.

### EXAMPLES

The following examples are put forth so as to provide those of ordinary skill in the art with a description of how the compositions and methods described herein may be used and evaluated and are intended to be purely exemplary of the invention and are not intended to limit the scope of what the inventors regard as their invention.

### Example 1. Use of Device for Cell Aggregate Encapsulation

A device as described herein containing a well plate (1) with a plurality of wells (2) and an indexed mold floor (3) with protrusions (4) is provided (see, e.g., FIG. 5). The device may contain a fluid tray (9), and a vessel (6) that includes a tube array housing (7) with storage tubes (8) (see, e.g., FIG. 10). The wells (2) may be sealed with gaskets (5).

A medium containing a biocompatible scaffold of fibrin and population of cell aggregates of stromal cells and parenchymal cells may be introduced into wells (2) of well plate (1) (see, e.g., FIG. 2). The medium may further contain a reinforcing agent, such as collagen. A polymerizing agent (e.g., thrombin) may be added to polymerize the fibrinogen and encapsulate the cell aggregates, e.g., in fibrin and collagen. Following encapsulation, the indexed mold floor (3) may be pushed into the wells (2) of well plate (1) to eject the grafts from each well (see, e.g., FIG. 3). The grafts may be collected in a storage tube (8), which contains a storage medium (see, e.g., FIG. 10). The grafts may be saved for subsequent use or implantation into a human subject.

### Example 2. Formation of Encapsulated Aggregates

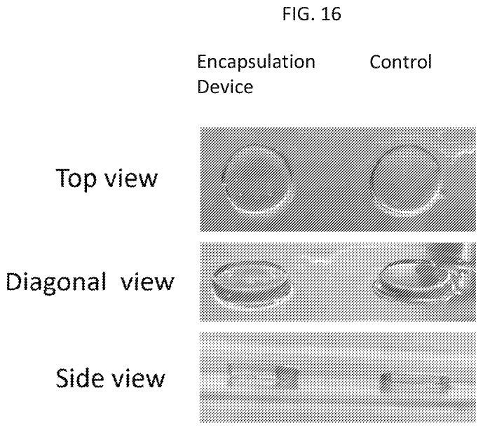

A prototype device was used to form encapsulated aggregates using a device and method as described herein. As shown in FIG. 16, encapsulated aggregates were formed using the encapsulation device.

### Other Embodiments

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. A device comprising:
(a) a well plate comprising one or more wells; and
(b) an indexed mold floor;
wherein the indexed mold floor comprises one or more protrusions that are configured to insert into the one or more wells and form a bottom surface for the one or more wells.

2. The device of claim 1, wherein the indexed mold floor is slidably connected to the well plate such that the one or more protrusions can move vertically with respect to the one or more wells.

3. The device of claim 1 or 2, wherein the indexed mold floor is rigid or flexible.

4. The device of any one of claims 1-3, wherein the one or more wells are cylindrical, oval, square, rectangular, pentagonal, hexagonal, heptagonal, or octagonal.

5. The device of any one of claims 1-4, wherein each well has a diameter of from 1 mm to 500 mm.

6. The device of any one of claims 1-5, wherein the device further comprises one or more gaskets, wherein each gasket is configured to form a seal between each protrusion and each well.

7. The device of claim 6, wherein the protrusion comprises the gasket.

8. The device of any one of claims 1-7, wherein the device further comprises a vessel connected to the well plate and configured to serve as a storage receptacle.

9. The device of claim 8, wherein the vessel comprises a tube array housing comprising one or more storage tubes.

10. The device of claim 9, wherein the one or more storage tubes are aligned with the one or more wells.

11. The device of claim 9 or 10, wherein each storage tube has a volume of from 1 cm³ to 1,000 cm³.

12. The device of any one of claims 1-11, wherein the well plate further comprises a fluid tray connected to the well plate and configured to prevent fluid overflow.

13. The device of claim 12, wherein the fluid tray has a height of from 0.1 mm to 100 mm above a surface of the well plate.

14. The device of claim 13, wherein the height of the fluid tray is from 0.1 mm to 10 mm above the surface of the well plate.

15. The device of any one of claims 1-14, further comprising a lid configured to cover the well plate.

16. The device of any one of claims 1-15, further comprising one or more sample identification elements.

17. The device of claim 16, wherein the one or more sample identification elements is present on the lid.

18. The device of any one of claims 1-17, further comprising a fastening element that secures the indexed mold floor to the well plate.

19. A method of encapsulating a population of aggregates comprising a first population of cells and a second population of cells comprising:
(a) providing the device of any one of claims 1-18;
(b) introducing a population of aggregates and a biocompatible scaffold into the one or more wells; and
(c) introducing a polymerizing agent into the one or more wells to polymerize the biocompatible scaffold, thereby encapsulating a population of aggregates.

20. The method of claim 19, wherein the biocompatible scaffold comprises fibrinogen.

21. The method of claim 19 or 20, wherein the biocompatible scaffold further comprises a reinforcing agent.

22. The method of claim 21, wherein the reinforcing agent comprises collagen, poly(ethylene glycol), polyvinylidene acetate (PVDA), polyvinylidene fluoride (PVDF), poly(lactic-co-glycolic) acid (PLGA), or poly (l-lactic acid) (PLLA).

23. The method of any one of claims 19-22, wherein the polymerizing agent comprises thrombin.

24. The method of any one of claims 19-23, wherein the device further comprises a vessel connected to the well plate and configured to serve as a storage receptacle.

25. The method of claim 24, wherein the vessel comprises a storage media.

26. The method of any one of claims 19-25, wherein the method further comprises raising the indexed mold floor to eject the encapsulated population of aggregates from the one or more wells.

27. The method of claim 26, wherein the method further comprises distributing the encapsulated population of aggregates from the one or more wells into the one or more storage tubes.

28. The method of claim 27, the encapsulated population of aggregates is distributed by gravity.

29. The method of any one of claims 19-28, wherein the first population of cells and/or the second population of cells are induced pluripotent (iPSC)-derived cells, engineered cells, primary cells, embryonic stem cells (ESC)-derived cells, or transdifferentiated cells.

30. The method of any one of claims 19-29, wherein the first population of cells comprises stromal cells and the second population of cells comprises parenchymal cells.

31. The method of claim 30, wherein the parenchymal cells are hepatocytes, pancreatic exocrine cells, myocytes, pancreatic endocrine cells, neurons, enterocytes, adipocytes, splenic cells, kidney cells, biliary cells, Kupffer cells, stellate cells, cardiac muscle cells, alveolar cells, bronchiolar cells, club cells, urothelial cells, mucous cells, parietal cells, chief cells, G cells, goblet cells, enteroendocrine cells, Paneth cells, M cells, tuft cells, glial cells, gall bladder cells, keratinocytes, melanocytes, Merkel cells, Langerhans cells, osteocytes, osteoclasts, esophageal cells, photoreceptor cells, or corneal epithelial cells.

32. The method of claim 31, wherein the hepatocytes are primary human hepatocytes.

33. The method of any one of claims 29-32, wherein the stromal cells are fibroblasts, endothelial cells, or pericytes.

34. The method of claim 33, wherein the fibroblasts are normal human dermal fibroblasts or neonatal foreskin fibroblasts.

35. The method of claim 34, wherein the fibroblasts are normal human dermal fibroblasts.

36. An encapsulated population of aggregates comprising a first population of cells and a second population of cells produced by the method of any one of claims 19-35.

37. The encapsulated population of aggregates of claim 36, wherein the first population of cells comprises stromal cells and the second population of cells comprises parenchymal cells.

38. The encapsulated population of aggregates of claim 36 or 37, wherein the encapsulated population of aggregates is suitable for implantation into a subject.

39. The encapsulated population of aggregates of claim 38, wherein the subject is a human.

40. A system comprising:
(a) a well plate comprising one or more wells; and
(b) an indexed mold floor;
wherein the indexed mold floor comprises one or more protrusions that are configured to insert into the one or more wells and form a bottom surface for the one or more wells.

41. The system of claim 40, further comprising a vessel.

42. The system of claim 41, wherein the vessel comprises a tube array housing comprising one or more storage tubes.

43. The system of claim 42, wherein the one or more storage tubes are aligned with the one or more wells.

44. A kit comprising:
(a) a well plate comprising one or more wells; and
(b) an indexed mold floor;
wherein the indexed mold floor comprises one or more protrusions that are configured to insert into the one or more wells and form a bottom surface for the one or more wells.

45. The kit of claim 44, further comprising a vessel.

46. The kit of claim 45, wherein the vessel comprises a tube array housing comprising one or more storage tubes.

47. The kit of claim 46, wherein the one or more storage tubes are aligned with the one or more wells.

48. The kit of any one of claims 44-47, further comprising one or more of a population of aggregates comprising a first population of cells and a second population of cells, a biocompatible scaffold, and a polymerizing agent.

49. The kit of claim 48, wherein the first population of cells comprises stromal cells and the second population of cells comprises parenchymal cells.
